(19) United States
(12) Patent Application Publication
(10) Pub. No.: US 2022/0282197 A1
(43) Pub. Date: Sep. 8, 2022

(54) Bioreactor Tile Including Fluidic Channels and an Optical Waveguide

(71) Applicant: IMEC VZW, Leuven (BE)

(72) Inventors: Candice M. Hovell, Titusville, FL (US); Jeremy Mares, Orlando, FL (US)

(21) Appl. No.: 17/653,070

(22) Filed: Mar. 1, 2022

Related U.S. Application Data
(60) Provisional application No. 63/157,654, filed on Mar. 6, 2021.

(51) Int. Cl. C12M 1/00; F21V 8/00; C12M 1/12; C12M 1/42; C12M 3/00

(52) U.S. Cl. CPC C12M 31/08; G02B 6/0078; C12M 23/34; C12M 25/00; C12M 47/10; C12M 35/08; C12M 23/44

(57) ABSTRACT

The present disclosure relates to bioreactor tiles including fluidic channels and optical waveguides. One example system includes a substrate having a first channel, a second channel, and a third channel defined therein. The three channels are separated from one another by partial wall structures. The system also includes an optical waveguide configured to receive illumination light at a first end of the optical waveguide; propagate the illumination light toward a second end of the optical waveguide; allow at least a portion of the illumination light to escape the optical waveguide from a first surface of the optical waveguide as the illumination light propagates toward the second end of the optical waveguide; and provide the portion of the illumination light that escapes the optical waveguide from the first surface to the second channel or the third channel.

### CROSS-REFERENCE TO RELATED APPLICATIONS

The present application claims priority to U.S. Provisional Patent Application No. 63/157,654, filed Mar. 6, 2021, which is hereby incorporated by reference in its entirety.

### BACKGROUND

Unless otherwise indicated herein, the materials described in this section are not prior art to the claims in this application and are not admitted to be prior art by inclusion in this section.

Currently, whole Cannabis sativa plants are grown, harvested, trimmed, dried, and processed via extraction to obtain useful compounds that are produced and stored in glandular trichomes. This process requires significant infrastructure, equipment, labor, and resources spread across multiple facilities. Further, this process may be susceptible to adverse effects from environmental conditions (e.g., changes in weather or contamination from heavy metals, pests, pesticides, fungal toxins, etc.).

Bioreactors are used in many contexts to grow or culture organisms (e.g., bacteria, plants, etc.). Such organisms may ultimately produce products that are harvested and used in various applications (e.g., pharmaceuticals, food production, etc.). Bioreactors may include light emitters, heaters, fluidic channels, etc. that support the environmental conditions necessary to sustain such organisms. For example, the pH, nutrient supply/concentration, temperature, etc. may all be monitored/adjusted within a bioreactor in order to mimic environmental conditions present in an organism's natural environment. Sample types of bioreactors include continuous stirred tank bioreactors, bubble column bioreactors, airlift bioreactors, fluidized bed bioreactors, packed bed bioreactors, and photo-bioreactors. Each of the various types may be associated with providing different environmental conditions and/or may be designed to support different types of organisms.

### SUMMARY

The specification and drawings disclose embodiments that relate to bioreactor tiles that include fluidic channels and optical waveguides.

In a first aspect, the disclosure describes a system. The system includes a substrate having a first channel, a second channel, and a third channel defined therein. The first channel is separated from the second channel by a first partial wall structure. The second channel is separated from the third channel by a second partial wall structure. The system also includes an optical waveguide. The optical waveguide is configured to receive illumination light at a first end of the optical waveguide. The optical waveguide is also configured to propagate the illumination light toward a second end of the optical waveguide. Further, the optical waveguide is configured to allow at least a portion of the illumination light to escape the optical waveguide from a first surface of the optical waveguide as the illumination light propagates toward the second end of the optical waveguide. Additionally, the optical waveguide is configured to provide the portion of the illumination light that escapes the optical waveguide from the first surface to the second channel or the third channel.

In a second aspect, the disclosure describes a system. The system includes a substrate having a first channel, a second channel, a third channel, a fourth channel, and a fifth channel defined therein. The first channel is separated from the second channel by a first partial wall structure. The second channel is separated from the third channel by a second partial wall structure. The first channel is separated from the fourth channel by a third partial wall structure. The fourth channel is separated from the fifth channel by a fourth partial wall structure. The system also includes a first optical waveguide. The first optical waveguide is configured to receive first illumination light at a first end of the first optical waveguide. The first optical waveguide is also configured to propagate the first illumination light toward a second end of the first optical waveguide. In addition, the first optical waveguide is configured to allow at least a portion of the first illumination light to escape the first optical waveguide from a first surface of the first optical waveguide as the first illumination light propagates toward the second end of the first optical waveguide. Further, the first optical waveguide is configured to provide the portion of the first illumination light that escapes the first optical waveguide from the first surface to the second channel or the third channel. Additionally, the system includes a second optical waveguide. The second optical waveguide is configured to receive second illumination light at a first end of the second optical waveguide. The second optical waveguide is also configured to propagate the second illumination light toward a second end of the second optical waveguide. Additionally, the second optical waveguide is configured to allow at least a portion of the second illumination light to escape the second optical waveguide from a first surface of the second optical waveguide as the second illumination light propagates toward the second end of the second optical waveguide. Further, the second optical waveguide is configured to provide the portion of the second illumination light that escapes the second optical waveguide from the first surface of the second optical waveguide to the fourth channel or the fifth channel.

In a third aspect, the disclosure describes a system. The system includes a plurality of substrates arranged into a vertical stack. Each of the substrates includes a first channel, a second channel, a third channel, a fourth channel, and a fifth channel defined therein. The first channel of each substrate is separated from the second channel of each substrate by a first partial wall structure. The second channel of each substrate is separated from the third channel of each substrate by a second partial wall structure. The first channel of each substrate is separated from the fourth channel of each substrate by a third partial wall structure. The fourth channel of each substrate is separated from the fifth channel of each substrate by a fourth partial wall structure. The system also includes a first optical waveguide. The first optical waveguide is configured to receive first illumination light at a first end of the first optical waveguide. The first optical waveguide is also configured to propagate the first illumination light toward a second end of the first optical waveguide. Additionally, the first optical waveguide is configured to allow at least a portion of the first illumination light to escape the first optical waveguide from a first surface of the first optical waveguide as the first illumination light propagates toward the second end of the first optical waveguide. Further, the first optical waveguide is configured to provide the portion of the first illumination light that escapes the first optical waveguide from the first surface to the second channels or the third channels defined within each of the plurality of substrates. In addition, the system includes a second optical waveguide. The second optical waveguide is configured to receive second illumination light at a first end of the second optical waveguide. The second optical waveguide is also configured to propagate the second illumination light toward a second end of the second optical waveguide. Additionally, the second optical waveguide is configured to allow at least a portion of the second illumination light to escape the second optical waveguide from a first surface of the second optical waveguide as the second illumination light propagates toward the second end of the second optical waveguide. Further, the second optical waveguide is configured to provide the portion of the second illumination light that escapes the second optical waveguide from the first surface of the second optical waveguide to the fourth channels or the fifth channels defined within each of the plurality of substrates.

In a fourth aspect, the disclosure describes a method. The method includes infusing a cellular precursor into a second channel defined within a substrate. The method also includes administering liquid nutrient media into a first channel defined within the substrate. The first channel is separated from the second channel by a first partial wall structure. Additionally, the method includes providing a first set of environmental conditions. The first set of environmental conditions results in a growth of cells within the cellular precursor. Further, the method includes providing a second set of environmental conditions. The second set of environmental conditions results in a production of trichomes by the cells within the cellular precursor. Providing the first set of environmental conditions or providing the second set of environmental conditions includes receiving, at a first end of an optical waveguide, illumination light. Providing the first set of environmental conditions or providing the second set of environmental conditions also includes propagating the illumination light toward a second end of the optical waveguide. Additionally, providing the first set of environmental conditions or providing the second set of environmental conditions includes allowing at least a portion of the illumination light to escape the optical waveguide from a first surface of the optical waveguide as the illumination light propagates toward the second end of the optical waveguide. Further, providing the first set of environmental conditions or providing the second set of environmental conditions includes providing the portion of the illumination light that escapes the optical waveguide from the first surface to the second channel or a third channel defined within the substrate. The second channel is separated from the third channel by a second partial wall structure. Still further, the method includes harvesting one or more of the produced trichomes or one or more chemical products contained within the produced trichomes.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE FIGURES

FIG. 1A is an isometric illustration of a bioreactor tile, according to example embodiments.

FIG. 1B is an isometric illustration of a bioreactor tile, according to example embodiments.

FIG. 1C is a side-view illustration of a bioreactor tile, according to example embodiments.

FIG. 1D is a front-view illustration of a bioreactor tile, according to example embodiments.

FIG. 2 is an isometric illustration of a bioreactor tile, according to example embodiments.

FIG. 3A is an isometric illustration of a bioreactor tile, according to example embodiments.

FIG. 3B is an isometric illustration of a bioreactor tile, according to example embodiments.

FIG. 3C is a front-view illustration of a bioreactor tile, according to example embodiments.

FIG. 4 is a front-view illustration of a multilayer bioreactor tile, according to example embodiments.

FIG. 5 is a schematic illustration of a bioreactor, according to example embodiments.

FIG. 6 is a schematic illustration of a computing device, according to example embodiments.

FIG. 7 is a flowchart illustrating a method, according to example embodiments.

### DETAILED DESCRIPTION

Example methods and systems are described herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. In addition, some of the illustrated elements may be combined or omitted. Similarly, an example embodiment may include elements that are not illustrated in the figures.

### I. Overview

Example embodiments relate to bioreactor tiles that include fluidic channels (e.g., microfluidic channels or non-microfluidic channels having a larger scale) and one or more optical waveguides. Such bioreactor tiles may be used to grow one or more types of cells. For example, the bioreactor tiles may support growth of plant cells (e.g., parenchymal plant cells) that produce one or more products (e.g., trichomes that contain desirable compounds). In some embodiments, for instance, example embodiments may be used to grow Cannabis sativa parenchymal cells that produce trichomes (e.g., glandular trichomes). Such trichomes may contain one or more organic compounds (e.g., cannabidiol), which can be harvested from the bioreactor tiles and used in a variety of applications. Using techniques described herein, the production of cannabidiol from Cannabis sativa parenchymal cells and trichomes can be performed in a way that results in: reduced exposure to outdoor environmental conditions; elimination of product contamination from heavy metals, pests, pesticides, fungal toxins, or products used in whole plant cultivation; a reduction in the total volume of solvents needed for product preparation; a simplification in the extraction process; a reduction in time, resources, and infrastructure required; an increased product purity; increased product properties (e.g., potency); and/or decreased carbon footprint for product isolation.

In some embodiments, the bioreactor tiles may include a series of channels (e.g., microfluidic channels defined within a substrate). For example, one channel may house the plant cells (e.g., within a gel). Another channel, parallel and adjacent to the channel housing the plant cells, may supply the plant cells with nutrients (e.g., in the form of liquid nutrient media). The nutrient channel may be supplied with nutrients using one or more pumps and/or based on a nutrient mixture provided from one or more tanks. For example, the nutrient channel may include one or more inlets and one or more outlets used to pass liquid nutrient media into and out of the nutrient channel. The type and concentration of nutrients supplied via this channel may depend on the type of plant cells housed within the plant cell channel. The plant cell channel and the nutrient media channel may be separated from one another by a partial wall structure. The partial wall structure may be of sufficient height so as to maintain separation between the components of the adjacent channels due to surface tension (i.e., the plant cells may stay in the plant cell channel based on surface tension above the partial wall structure and the liquid nutrient media may stay in the nutrient channel based on surface tension above the partial wall structure). However, in addition to having a sufficient height so as to maintain channel separation, the partial wall structure may nonetheless still allow the plant cells within the plant cell channel to retrieve the nutrients from the nutrient channel.

Additionally, the bioreactor tiles may include a product channel (e.g., a trichome channel). The product channel may be positioned parallel and adjacent to the plant cell channel on an opposite side of the plant cell channel from the nutrient channel. The product channel and the plant channel may also be separated by a partial wall structure that maintains component separation between the adjacent channels using surface tension (e.g., as viscous forces dominate over convective forces based on the size of the channels), but still allows interaction between the adjacent channels. Further, the product channel may include one or more inlets and one or more outlets (e.g., used to pass air and/or extractant into and out of the product channel). Once the plant cells within the plant cell channel have matured to a sufficient state, the plant cells may begin to produce one or more products (e.g., one or more chemical products). For example, in the case of Cannabis sativa cells, once the Cannabis sativa cells have sufficiently matured they will begin to produce trichomes (i.e., appendage structures that grow from the plant cells). The trichomes may grow out of the plant channel and extend into the product channel. Further, the trichomes may contain cannabidiol (e.g., cannabidiol oil), which is a widely used pharmaceutical substance.

Upon the plant cells beginning to produce trichomes, the bioreactor tile may provide an environment that further encourages trichome growth. In some embodiments, this may include providing the same environmental conditions that were provided by the bioreactor tile during the plant cell growth stage. However, in some embodiments, upon the initiation of the trichome phase, the environmental conditions supplied by the bioreactor tile may change (e.g., the type and/or concentration of the nutrients in the liquid nutrient media might change, the lighting conditions supplied to the trichomes/the plant cells may change, the temperature and/or pH may change, etc.). Once the trichomes are mature (e.g., when the concentration of monoterpenes is substantially higher than the concentration of sesquiterpenes within the trichomes), the trichomes and/or the products within the trichomes (e.g., cannabidiol) may be harvested. Harvesting the trichomes may involve flushing the product channel with extractant (e.g., low-temperature extractant) to shear trichome heads from the trichome bodies and/or to shear the trichomes from the underlying plant cells and then collecting the extractant/trichome mixture.

In some embodiments, there may also be an air inlet/outlet to supply the plant cells within the plant cell channel and/or the trichomes within the trichome channel with sufficient air (e.g., for photosynthesis, respiration, and/or to provide mechanical shear to the trichomes). Such an airflow may occur within one or more of the channels. For example, the air may be provided via the trichome channel. Prior to and during trichome growth, because the trichome channel is adjacent to and partially connected to the plant cell channel, the trichomes in the trichome channel and the plant cells in the plant cell channel can exchange gases with air flowing through the trichome channel. Such airflow may be generated by one or more pumps and/or based on an air mixture provided from one or more tanks. Additionally or alternatively, airflows may also be established within the plant cell channel or the nutrient channel.

In addition to nutrients and airflow, the plant cells (e.g., as well as trichomes) may use light to grow (e.g., to perform photosynthesis). This light may be supplied using one or more optical waveguides (i.e., light pipes) of the bioreactor tile. The optical waveguide(s) may run parallel to the longitudinal direction of the plant cell channel and/or the product channel. Additionally, the optical waveguide(s) may be positioned above the plant cell channel and/or the product channel. In such embodiments, the optical waveguide(s) may receive illumination light (e.g., emitted by a light-emitter diode (LED) or other light emitter) at a first end and then provide the illumination light to the plant cells (e.g., within the plant cell channel) and/or the trichomes (e.g., within the product channel) via a bottom surface of the optical waveguide. In some embodiments, the optical waveguide may be tapered (e.g., along a vertical direction) and/or may include one or more surface features (e.g., diffractive features, longitudinal striations, lateral striations, isotropic striations, pits, etc.) that permit the illumination light to escape the optical waveguide along the bottom surface of the optical waveguide as the illumination light propagates from the first end of the optical waveguide toward a second end of the optical waveguide.

Alternatively, in some embodiments, the optical waveguide(s) may be positioned on a surface of the substrate that is adjacent to the product channel. For example, the optical waveguide(s) may be surface-integrated optical waveguide(s) (e.g., fabricated on the surface of the substrate using microfabrication techniques). In such embodiments, the optical waveguide(s) may receive illumination light (e.g., emitted by a LED or other light emitter) at a first end and then provide the illumination light to the plant cells (e.g., within the plant cell channel) and/or the trichomes (e.g., within the product channel) from a side surface of the optical waveguide. As with the suspended/cantilevered optical waveguide positioning described above, such surface-integrated optical waveguides may be tapered (e.g., along a vertical direction) and/or may include one or more surface features that permit the illumination light to escape the optical waveguide along the side surface of the optical waveguide as the illumination light propagates from the first end of the optical waveguide toward a second end of the optical waveguide.

Regardless of the position(s) of the optical waveguide(s) relative to the product channel or the plant cell channel, the optical waveguide(s) may be configured to distribute the illumination light in various intensities along the lengths of the plant cell channel and/or the product channel. For example, in some embodiments, the optical waveguide may provide the illumination light to the product channel and/or the plant cell channel in a substantially uniform intensity along the length of the respective channel (e.g., +/−0.1% variation along the length of the respective channel, +/−1.0% variation along the length of the respective channel, +/−5.0% variation along the length of the respective channel, +/−10.0% variation along the length of the respective channel, or +/−15% variation along the length of the respective channel). In other embodiments, though, the optical waveguide may provide the illumination light in a manner that is intentionally varied along the length of the respective channel.

While a three-channel arrangement (e.g., an arrangement having a single nutrient channel, a single plant cell channel, and a single product channel, which may be referred to herein as a “single-sided arrangement”) has been described above, it is understood that other arrangements are also possible and are contemplated herein. For example, in some embodiments, a single nutrient channel may supply liquid nutrient media to two plant cell channel/product channel pairs. In such embodiments, the nutrient channel may be flanked, on both sides, by plant cell channels (e.g., each of the plant cell channels separated from the nutrient channel by a partial wall structure). Further, each of the two plant cell channels may be flanked (on a side of the plant cell channel opposite from the side of the plant cell channel that is adjacent to the nutrient channel) by a respective product channel. This alternate arrangement may be referred to herein as a “double-sided arrangement.” It is understood that numerous other arrangements are also possible (e.g., four-channel arrangements, five-channel arrangements, six-channel arrangements, seven-channel arrangements, eight-channel arrangements, nine-channel arrangements, ten-channel arrangements, etc.). For example, some embodiments may include a seven-channel arrangement (e.g., a double-sided, seven-channel arrangement having an air channel, flanked on both sides by channels configured to house suspensions of plant cells, each of those channels being flanked on outer sides by gel nutrient media channels, each of those channels being flanked on outer sides by liquid nutrient media channels).

Further, while a bioreactor tile with only one single-sided arrangement of channels defined within the substrate is described above, it is understood that many sets of channels may be defined within the same substrate. For example, a plurality of single-sided arrangements of channels may be defined adjacently to one another within the substrate. Likewise, a plurality of double-sided arrangements of channels may be defined adjacently to one another within the substrate. In still other embodiments, a mixture of single-sided channel arrangements and double-sided channel arrangements may all be defined within a single substrate. Such arrangements having a plurality of sets of channels may provide for higher throughput (e.g., a larger production of product, such as cannabidiol oil, per unit time or per unit area).

Additionally, while a bioreactor tile having only a single substrate with channels defined therein (referred to herein as a “single-layer arrangement”) was described above, it is understood that other arrangements are also possible. For example, a “multi-layer arrangement” having multiple substrates all stacked on top of one another and each with one or more sets of channels defined therein is also contemplated herein. In such a multi-layer arrangement, there may be respective optical waveguides for each layer that only supply light to the channels in their respective layer. Alternatively, there may only be a single set of optical waveguides that supply light to all the channels in all of the layers. For example, the arrangements of channels defined in each of the substrates of each of the layers may be aligned vertically with one another. Then, positioned above these vertically aligned arrangements, a single optical waveguide could provide light to all the channels in the vertically aligned arrangements. Such embodiments may be used when the substrates and/or the contents of the channels (e.g., the liquid nutrient media, the plant cells in a gel matrix, etc.) are substantially transparent and/or translucent. Such multi-layer arrangements may provide for higher throughput (e.g., a larger production of product, such as cannabidiol oil, per unit time or per unit volume).

The bioreactor tiles described above may be designed to readily scale with desired production output. In some embodiments, a plurality of bioreactor tiles (e.g., two bioreactor tiles, three bioreactor tiles, four bioreactor tiles, five bioreactor tiles, ten bioreactor tiles, twenty bioreactor tiles, thirty bioreactor tiles, forty bioreactor tiles, fifty bioreactor tiles, one hundred bioreactor tiles, two hundred bioreactor tiles, three hundred bioreactor tiles, four hundred bioreactor tiles, five hundred bioreactor tiles, one thousand bioreactor tiles, etc.) may be slotted into a single bioreactor. For example, a bioreactor may include a cabinet the supports a number of bioreactor tiles. Each of the bioreactor tiles may be slotted into a different shelf of the cabinet. As such, the cabinet with the associated bioreactor tiles may be referred to herein as a “multi-tiled bioreactor.” The cabinet may also include additional components used to monitor and support the bioreactor tiles. For example, the cabinet may include one or more illumination sources (e.g., LEDs or other light emitters) that connect to the optical waveguide(s) of each of the bioreactor tiles in order to provide illumination light to the plant cell channels and/or the product channels via the optical waveguide(s). The illumination sources may be connected to a power supply of the cabinet, for example. Because the illumination sources are components of the cabinet, rather than the individual bioreactor tiles, any heat generated by the illumination sources may be produced in a region of the cabinet that is not so near to the plant cells and/or the trichomes so as to adversely impact the temperature near the plant cells/trichomes.

In addition to illumination sources, the cabinet may also include one or more tanks used to store nutrients, products, cellular precursors, air, gels, etc. for use with the bioreactor tiles; one or more pumps used to transport nutrients, products, cellular precursors, air, gels, etc. to and/or from the bioreactor tiles; and/or one or more tubes/pipes through which nutrients, products, cellular precursors, air, gels, etc. are transported to and/or from the bioreactor tiles. Additionally, the cabinet may include one or more heating devices or cooling devices used to maintain an environmental temperature throughout the cabinet or in specific locations of the cabinet. Further, the cabinet may include one or more devices used to monitor a state of one or more components of the cabinet. For example, the cabinet may include one or more sensors used to monitor a temperature inside various regions of the cabinet, one or more sensors used to monitor a humidity inside various regions of the cabinet, one or more sensors used to monitor an air pressure inside various regions of the cabinet, one or more sensors used to monitor concentration of nutrients within the nutrient channel, one or more sensors used to monitor light intensity within various regions of the cabinet, one or more sensors used to monitor pH in various channels, one or more sensors used to monitor flow rate of air through one or more tubes or channels, or one or more sensors used to sense an amount of nutrient, product, air, etc. within a tank in the cabinet.

Still further, in order to monitor the state of the plant cells in the plant cell channel and/or the trichomes in the product channel, each individual bioreactor tile or the entire cabinet may also include a camera or other optical imaging device (e.g., fluorescent microscope) configured to capture images or other optical data (depending on the imaging modality used) associated with the plant cell channels and/or the trichomes. Based on the captured images or other captured optical data, a computing device (e.g., a bioreactor controller) executing one or more instructions (e.g., stored within a memory, such as a hard disk) may make control determinations for the bioreactor. For example, the computing device may determine when to provide certain nutrient combinations to the nutrient channels of the bioreactor tiles, the temperature at which to maintain certain regions of the cabinet (e.g., to promote plant cell growth or trichome growth), when to harvest the products, what light intensity/illumination schedule to use to illuminate the plant cell channels or the product channels. As such, the computing device may be connected to one or more sensors of the cabinet, as well as one or more heating devices, one or more cooling devices, one or more power supplies, one or more pumps, one or more illumination sources, etc. of the cabinet. In some embodiments, the computing device may be configured to communicate with one or more remote computing devices (e.g., over BLUETOOTH®, the public Internet, etc.) to externally provide one or more statuses of the bioreactor (e.g., for remote monitoring/analysis).

Further, a plurality of such multi-tiled bioreactors may be used simultaneously within a single production facility. For example, a climate-controlled warehouse having a number of bioreactors that each include a plurality of bioreactor tiles may be used to produce large product quantities (e.g., tens or hundreds of kg of crude cannabidiol oil per trichome life-cycle). The production facility may include a central monitoring computing device that monitors data from each of the individual multi-tiled bioreactors to make control decisions across the entire facility. Additionally or alternatively, an off-site computing device may monitor the status of the bioreactors within the production facility.

In some embodiments, each of the bioreactor tiles in a single bioreactor may be used to grow/harvest the same product. Alternatively, in some embodiments, different bioreactor tiles may be used to grow/harvest different products. Likewise, in some embodiments, each of the multi-tiled bioreactors in a single production facility may be used to grow/harvest the same product. Alternatively, in some embodiments, different bioreactors in a single production facility may be used to grow/harvest different products. Further, while Cannabis sativa is described throughout as an example plant cell that could be grown to produce trichomes containing cannabidiol using the bioreactor tiles described herein, it is understood that other types of cells and/or other products are also possible (e.g., both plant cells that produce trichomes and those that do not). For example, other plant cells that produce trichomes and could be grown using the techniques described herein include Artemisia annua cells, Chrysanthemum cinerariifolium cells, Chrysanthemum coccineum cells, Gossypium hirsutum cells, Gossypium barbadense cells, Gossypium arboreum cells, Gossypium herbaceum cells, Lavandula angustifolia cells, Arabidopsis thaliana cells, Mentha x piperita cells, and Mentha haplocalyx cells. Such plant cells could produce products that include tetrahydrocannabinol, artemisinin, pyrethrum, camphor, glucosinolate, linalool, linalyl acetate, menthol, or peppermint camphor. Still further, the techniques and devices described herein could be used to grow other types of cells (i.e., non-plant cells) and produce products (e.g., trichome products). For example, algae cells, lichen cells, and protist cells could be grown.

### II. Example Systems

The following description and accompanying drawings will elucidate features of various example embodiments. The embodiments provided are by way of example, and are not intended to be limiting. As such, the dimensions of the drawings are not necessarily to scale.

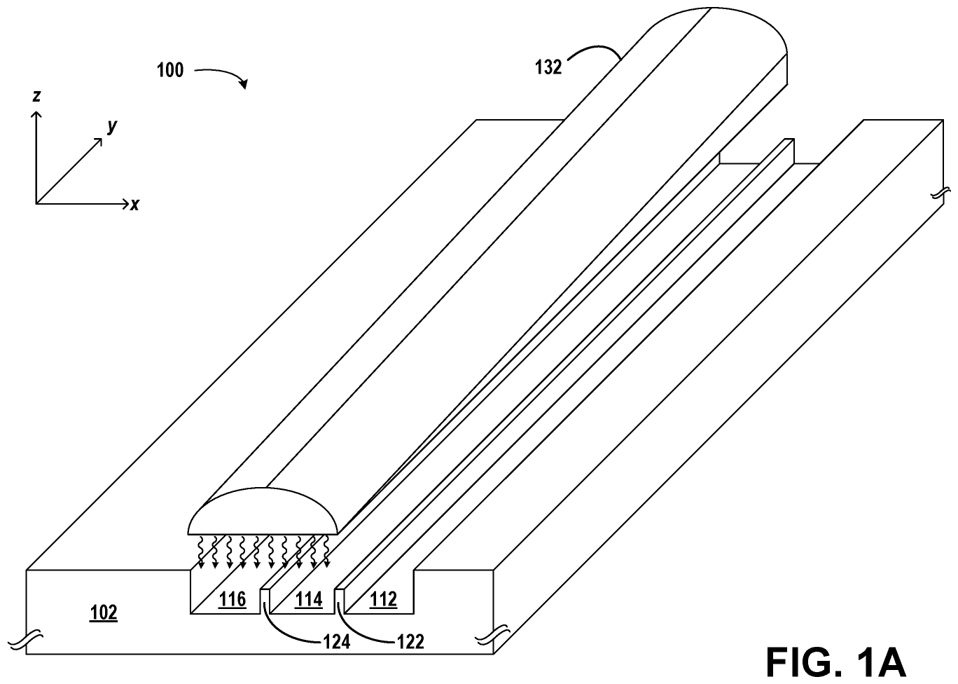

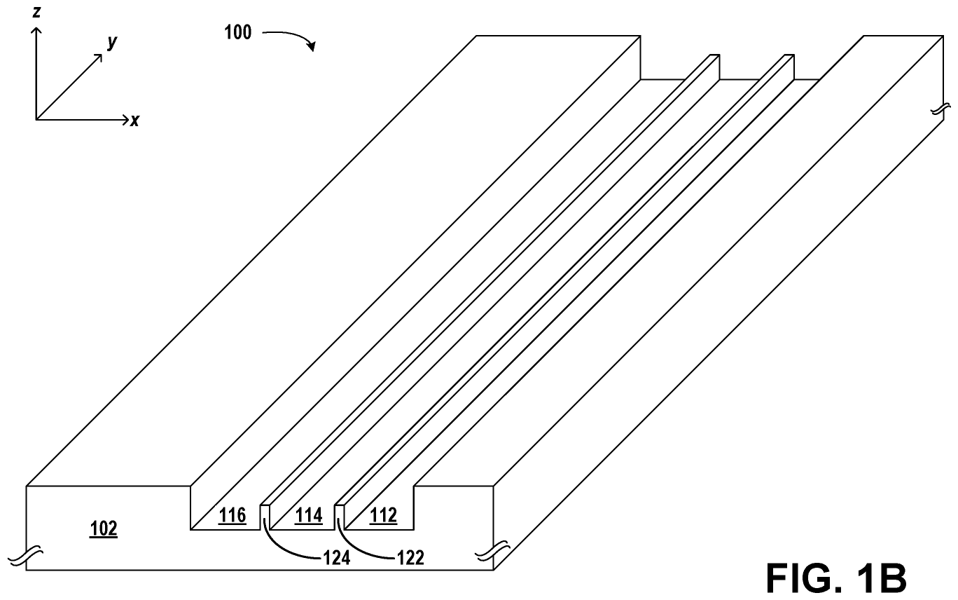

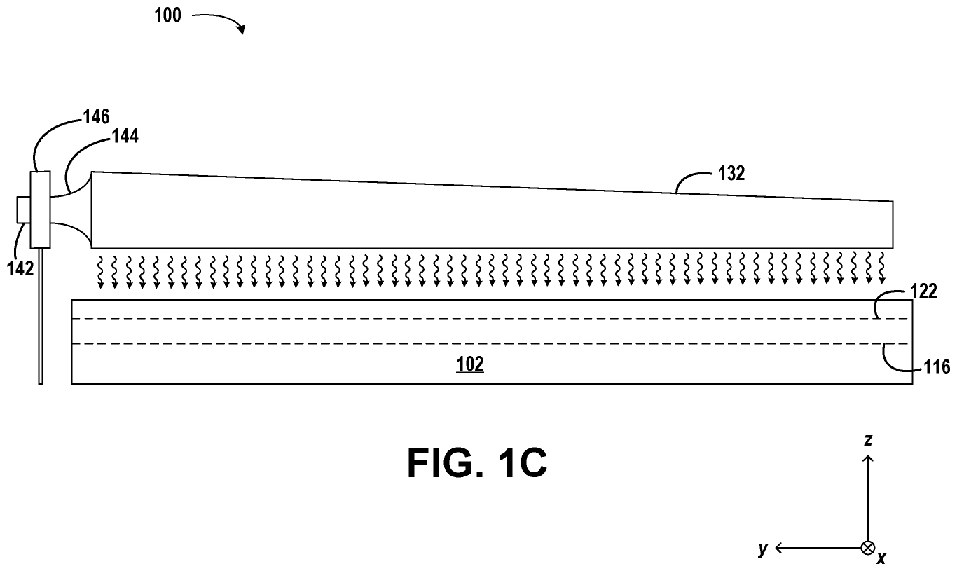

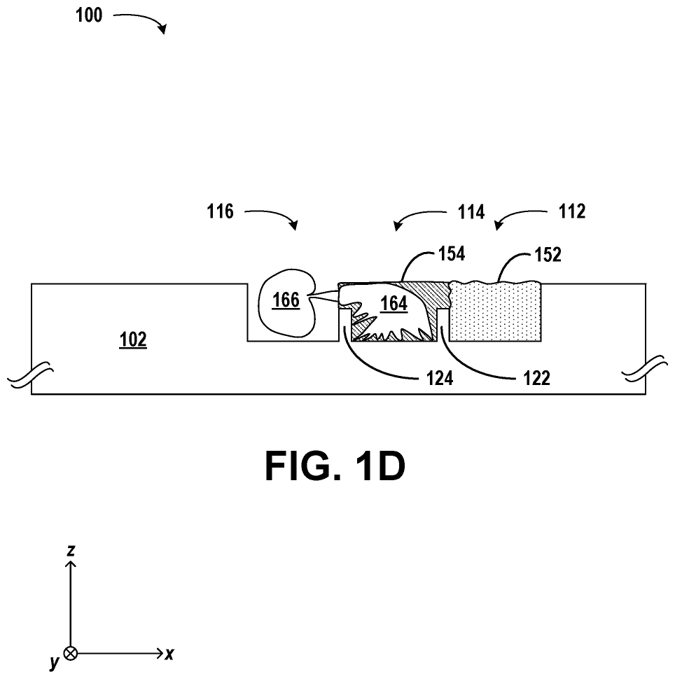

FIG. 1A is an isometric illustration of a bioreactor tile 100, according to example embodiments. The bioreactor tile 100 may include a substrate 102 having a first channel 112 (i.e., a nutrient channel 112), a second channel 114 (i.e., a plant cell channel 114), and a third channel 116 (i.e., product channel 116) defined therein. The first channel 112 may be separated from the second channel 114 by a first partial wall structure 122 and the second channel 114 may be separated from third channel 116 by a second partial wall structure 124. The bioreactor tile 100 may also include an optical waveguide 132. The arrangement of the bioreactor tile 100 of FIG. 1A may be referred to as a single-sided arrangement. FIG. 1B is an isometric illustration of the same bioreactor tile 100 as FIG. 1A, with the optical waveguide 132 removed from the illustration to make the underlying structures of the first channel 112, the second channel 114, the third channel 116, the first partial wall structure 122, and the second partial wall structure 124 more clearly visible.

The substrate 102 may include a surface into which one or more structures (e.g., the first channel 112, the second channel 114, the third channel 116, the first partial wall structure 122, and/or the second partial wall structure 124) are defined (e.g., via etching) and/or onto which one or more structures are deposited (e.g., via chemical vapor deposition (CVD)). For example, the substrate 102 may include a transparent polymer (e.g., polydimethylsiloxane (PDMS)), glass (e.g., fused silica, borosilicate, soda-lime glass, etc.), thermoplastics (e.g., poly(methyl methacrylate) (PMMA), acrylic, polycarbonate, optically clear polyvinyl chloride (PVC), polyethylene terephthalate glycol (PETG), etc.), polylactic acid (PLA), thermoset polymers, and/or a Si wafer that may be processed (e.g., using microfabrication techniques, additive manufacturing techniques, or molding techniques) to define structures into the substrate 102. Other substrates are also possible and are contemplated herein. As illustrated by the discontinuous lines oriented along a z-direction of the substrate, in some embodiments, a thickness of the substrate (e.g., between 500 μm and 3,000 μm) may be considerably larger than a thickness of the channels 112, 114, 116 (e.g., between 250 μm and 2,000 μm) and/or the partial wall structures 122, 124 (e.g., between 250 μm and 1,500 μm).

The first channel 112, the second channel 114, and the third channel 116 may run substantially parallel to one another (e.g., parallel to the y-axis), as illustrated in FIGS. 1A and 1B. In some embodiments, the first channel 112, the second channel 114, and the third channel 116 may be fluidic channels (e.g., microfluidic channels or larger-scale channels). For example, the first channel 112, the second channel 114, and/or the third channel 116 may be: between 250 μm and 2,000 μm in width (x-dimension in FIG. 1A), between 10 cm and 100 cm in length (y-dimension in FIG. 1A), and between 500 μm and 2,000 μm in height (z-dimension in FIG. 1A). In some embodiments, the first channel 112, the second channel 114, and the third channel 116 may be configured to house different substances of the bioreactor tile 100. For example, as shown and described further below with reference to FIG. 1D, the first channel 112 may be configured to house nutrients (e.g., a liquid nutrient media), the second channel 114 may be configured to house plant cells (e.g., within a gel from a cellular precursor), and the third channel 116 may be configured to house products (e.g., trichomes that are produced by the plant cells and are growing in an air environment within the third channel 116).

Further, as illustrated and described above, the first channel 112 may be separated from the second channel 114 by the first partial wall structure 122. In some embodiments, the first partial wall structure 122 may be between 250 μm and 1,000 μm in width (x-dimension in FIG. 1A), between 10 cm and 100 cm in length (y-dimension in FIG. 1A), and between 250 μm and 1,500 μm in height (z-dimension in FIG. 1A). The first partial wall structure 122 may serve to separate the two channels 112, 114 from one another while still permitting some contact between the two channels 112, 114. For example, surface tension present in a fluid in the first channel 112, a fluid in the second channel 114, or both might (along with the first partial wall structure 122 itself) form an effective barrier between the first channel 112 and the second channel 114. However, in a region above the first partial wall structure 122, the contents/fluid in the first channel 112 may be in contact with the contents/fluid in the second channel 114, which may allow substances (e.g., nutrients) within the first channel 112 to be transported to the second channel 114 (e.g., via diffusion). Similarly, the second partial wall structure 124 may serve to separate the second channel 114 from the third channel 116 while still permitting some contact between the two channels 114, 116 (e.g., thereby allowing substances, such as trichomes within the second channel 114 to grow/extend into the third channel 116). Like the first partial wall structure 122, the second partial wall structure 124 may be between 250 μm and 1,000 μm in width (x-dimension in FIG. 1A), between 10 cm and 100 cm in length (y-dimension in FIG. 1A), and between 250 μm and 1,500 μm in height (z-dimension in FIG. 1A).

While the first channel 112, the second channel 114, and the third channel 116 are illustrated in FIGS. 1A and 1B as having roughly the same dimensions (e.g., same width, depth, and length), it is understood that other embodiments are also possible. For example, one of the channels 112, 114, 116 may be wider and/or deeper than the other two channels to accommodate additional volume (e.g., the second channel 114 may be deeper than the other two channels, shallower than the other two channels, wider than the other two channels, or narrower than the other two channels). Additionally or alternatively, one of the channels 112, 114, 116 may be longer than the other two channels so as to connect it to an inlet that is at a different location within the bioreactor tile 100 than the inlets of the other two channels. Similarly, while the first partial wall structure 122 and the second partial wall structure 124 are illustrated in FIGS. 1A and 1B as having roughly the same dimensions (e.g., same width, height, and length), it is understood that other embodiments are also possible. For example, one of the partial wall structures 122, 124 may be narrower and/or shorter than the other partial wall structure (e.g., to promote additional fluid interaction between the channels adjacent to that partial wall structure). Further, in some embodiments, one or both of the partial wall structures 122, 124 may have excised portions (e.g., removed sections along the y-axis illustrated in FIGS. 1A and 1B) that further promote fluid interaction between channels adjacent to that partial wall structure.

The first channel 112, the second channel 114, the third channel 116, the first partial wall structure 122, and/or the second partial wall structure 124 may be fabricated (e.g., may be defined within the substrate 102) using a variety of fabrication techniques (e.g., microfabrication techniques performed within a cleanroom setting or additive manufacturing techniques). For example, fabricating the bioreactor tile 100 may include providing the substrate 102 (e.g., a PMMA wafer or an acrylic wafer) and performing one or more etching steps. Such etching steps may include selective etching steps, chemical etching steps, wet etching steps, dry etching steps, etc. Further, in embodiments where selective etching steps are performed, one or more photolithography processing steps (e.g., to define one or more masks used to perform the selective etching steps) may also be performed. The etch depths used to define the channels 112, 114, 116 may be different than the etch depths used to define the partial wall structures 122, 124, for example. Additionally or alternatively, in some embodiments, one or more planarization processes may be performed (e.g., a chemical-mechanical polishing (CMP) process) during the fabrication of the bioreactor tile 100. Still further, in some embodiments, a plasma bonding process and/or a dry bonding process may be performed during the fabrication of the bioreactor tile 100. It is understood that, while microfabrication techniques can be used in fabrication, various embodiments may also incorporate some (e.g., may incorporate exclusively) fabrication techniques that are of such a size/scale that such microfabrication techniques are not necessary.

Returning to FIG. 1A, the optical waveguide 132 may be fabricated along with the substrate 102 and the channels 112, 114, 116/partial wall structures 122, 124 defined therein. Alternatively, the optical waveguide 132 may be fabricated separately from the substrate 102 and the channels 112, 114, 116/partial wall structures 122, 124 defined therein and later attached to the substrate 102 and the channels 112, 114, 116/partial wall structures 122, 124 defined therein to form the bioreactor tile 100. The optical waveguide 132 may receive electromagnetic waves (e.g., illumination light) at a first end (e.g., an input end) of the optical waveguide 132 (e.g., via an optical coupling to the optical waveguide 132, as shown and described with reference to FIG. 1C). For example, as illustrated in FIG. 1A, the optical waveguide 132 may receive light at an end of the optical waveguide 132 having the greatest y-position (as illustrated by the axes in FIG. 1A). Upon receiving the electromagnetic waves, the electromagnetic waves may propagate along the optical waveguide 132 toward a second end of the optical waveguide 132 (e.g., an end of the optical waveguide 132 having the lowest y-position, as illustrated in FIG. 1A).

Propagation of electromagnetic waves along the optical waveguide 132 may occur, at least partially, due to total internal reflection from one or more surfaces of the optical waveguide 132. For example, in some embodiments, the optical waveguide 132 may be fabricated from SiO₂. As such, there may be a mismatch between the material of the optical waveguide 132 and the surrounding environment of the optical waveguide 132 (e.g., air). This material mismatch may also correspond to a mismatch in relative dielectric constants (εᵣ)/refractive indices (η). For example, in embodiments where the optical waveguide 132 is fabricated from SiO₂ and the surrounding environment is air, the mismatch of relative dielectric constants may be ˜3.9 (εᵣ of SiO₂) to ˜1 (εᵣ of air). It is understood that these values are given solely as examples, and that other materials may be used and/or the materials listed may have different relative dielectric constants depending on the wavelength of electromagnetic signal propagating within the materials. As a result of Snell's law, such a material mismatch may lead to total internal reflection for a specified range of incidence angles of the electromagnetic waves. It is understood that other materials besides SiO₂ for the optical waveguide 132 are also possible and are contemplated herein (e.g., PMMA, polycarbonate, polyetherimide, ZnO, high-index glasses). Further, while the optical waveguide 132 of FIGS. 1A and 1C may not have an optical coating, it is understood that, in other embodiments, the optical waveguide 132 may have one or more portions coated with an optical coating (e.g., a reflective optical coating) to prevent illumination light from escaping the optical waveguide 132 at the coated regions.

Further, as illustrated in FIG. 1A, the optical waveguide 132 may be positioned above (e.g., suspended above, mounted above, cantilevered above, etc.) the second channel 114 and the third channel 116. As such, the optical waveguide 132 may provide illumination light (e.g., illumination light received at the first end of the optical waveguide 132) to the second channel 114 or the third channel 116 (e.g., in order to provide conditions conducive to the growth of plant cells in the second channel 114 and/or the growth of trichome cells in the third channel 116). This is illustrated by the light rays emitted from the bottom of the optical waveguide 132 in FIG. 1A. Providing the illumination light to the second channel 114 and/or the third channel 116 may occur as a result of one or more surface features present on an underside (e.g., a flat bottom surface) of the optical waveguide 132 that allow illumination light to escape the optical waveguide 132 (e.g., that interrupt total internal reflection) as the illumination light propagates in the optical waveguide 132. For example, the bottom surface of the optical waveguide 132 may include diffractive features, longitudinal striations (e.g., running parallel to the y-axis illustrated in FIG. 1A), lateral striations (e.g., running parallel to the x-axis illustrated in FIG. 1A), or pits.

In addition, one or more surfaces of the optical waveguide 132 may be tapered. For example, as illustrated in FIG. 1A, a top surface of the optical waveguide 132 (e.g., a surface of the optical waveguide 132 having the greatest z-position) may be tapered (e.g., from a first end of the optical waveguide 132 to a second end of the optical waveguide 132). Such a tapering may provide for a relatively uniform intensity of illumination light provided to the second channel 114 and/or the third channel 116 by the optical waveguide 132. In other embodiments, other shapes of optical waveguide 132 may be used. For example, the intensity of the illumination light provided to the second channel 114 and/or the third channel 116 may substantially vary along the length (e.g., y-position) of the second channel 114 and/or the third channel 116.

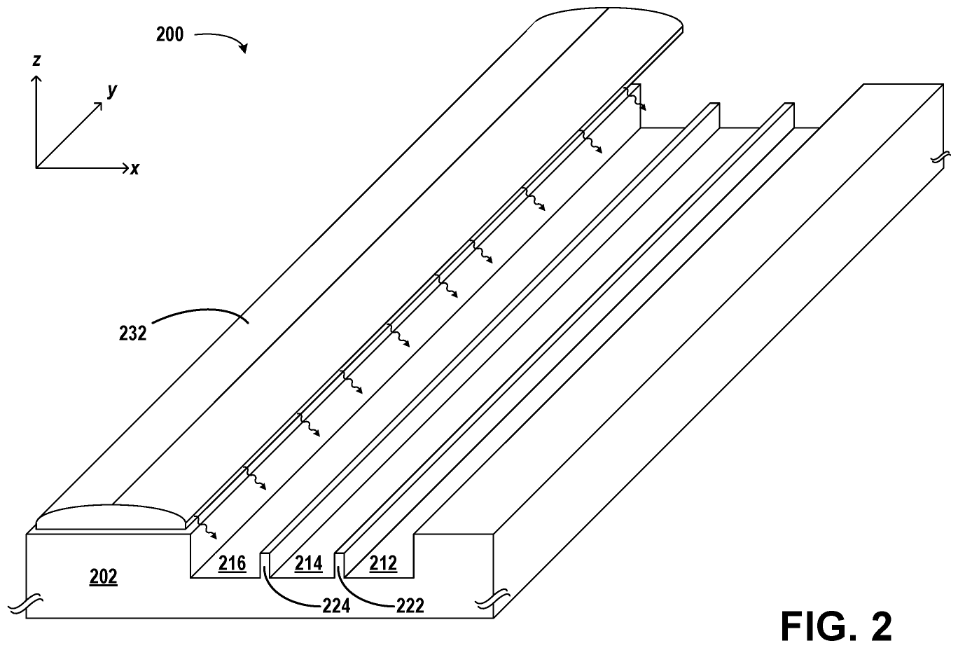

It is understood that other positions of the optical waveguide 132 are also possible and are contemplated herein. For example, the optical waveguide 132 may be positioned adjacent to, below, or otherwise near one or more of the channels 112, 114, 116. It is also understood that other numbers of optical waveguides within the bioreactor tile 100 (e.g., two optical waveguides, three optical waveguides, four optical waveguides, etc.) are also possible and are contemplated herein. Such alternative possibilities are further shown and described with reference to FIGS. 2, 3A, and 4.

FIG. 1C is a side-view illustration (i.e., parallel to the y-z plane, as illustrated) of the bioreactor tile 100 illustrated and described with reference to FIGS. 1A and 1B. As illustrated by dashed lines in FIG. 1C, the third channel 116 and the second partial wall structure 124 may be defined within and obscured by a side of the substrate 102. As further illustrated in FIG. 1C, the optical waveguide 132 of the bioreactor tile 100 may also include a mixing region 142 and a coupling region 144. The mixing region 142 of the optical waveguide 132 may receive illumination light from an illumination source (e.g., a LED or other light emitter that is part of a multi-tile bioreactor, as described above). Further, the bioreactor tile 100 may include a mounting structure 146 used to support the optical waveguide 132. The mixing region 142, the coupling region 144, and the mounting structure 146 may each be components present in FIG. 1A, for example, but that are obscured by other components based on the isometric view of FIG. 1A.

The mixing region 142 may be configured to homogenize modes or wavelengths present within the illumination light received at the first end of the optical waveguide. For example, the mixing region 142 may combine illumination light having different modes into a single mode (e.g., by modifying modes that correspond to modes other than a predetermined mode so that they match the predetermined mode). Further, in some embodiments, the mixing region 142 may select only a predefined range of wavelengths from a group of input wavelengths (e.g., from illumination light coming from multiple illumination sources). For example, the mixing region 142 may attenuate, reflect, or otherwise reject light having wavelengths outside of the predefined range of wavelengths, thereby preventing wavelengths outside of the predefined range from propagating through the coupling region 144 and/or the remainder of the optical waveguide 132. In some embodiments, the mixing region 142 may be fabricated of the same material as the rest of the optical waveguide 132 (e.g., as the coupling region 144). Alternatively, though, the mixing region 142 may be fabricated from a different material than the rest of the optical waveguide 132.

The coupling region 144 may be configured to couple illumination light (e.g., the homogenized illumination light from the mixing region 142) into a main body of the optical waveguide. As such, the coupling region 144 may be tapered from the mixing region 142 to the main body of the optical waveguide 132. In various embodiments, the taper of the coupling region 144 may have various shapes/lengths (i.e., y-dimensions). For example, the length of the taper of the coupling region 144 may be based on a predetermined range of illumination wavelengths to be used with the optical waveguide 132 (e.g., to be received by the optical waveguide 132 at the mixing region 142 and/or to be provided from the optical waveguide 132 to the second channel 114 and/or the third channel 116). In some embodiments, the coupling region 144 may be fabricated of the same material as the main body of the optical waveguide 132. Alternatively, though, the coupling region 144 may be fabricated from a different material than the rest of the optical waveguide 132.

The mounting structure 146 may be a structure of the bioreactor tile 100 to which one or more optical waveguides (e.g., the optical waveguide 132) is attached/mounted. For example, as illustrated in FIG. 1C, the mounting structure may include a slot through which a portion of the optical waveguide 132 (e.g., the mixing region 142 and/or the coupling region 144) is passed/on which the optical waveguide 132 rests. The optical waveguide 132 may extend from the slot over the substrate 102 (e.g., may be cantilevered over the substrate) as illustrated in FIG. 1C. In some embodiments, the mounting structure 146 may be defined within or attached to the substrate 102. In other embodiments, the mounting structure 146 may be freestanding within the bioreactor tile 100. In some embodiments, there may be multiple mounting structures associated with a single optical waveguide. For example, there may be a first mounting structure positioned at the first end of the substrate to support the optical waveguide from the first end. Additionally, there may be a second mounting structure positioned at a second end of the substrate (e.g., an opposite end of the substrate from the first mounting structure) and configured to support the optical waveguide from the opposite end. In still other embodiments, there may be more than two mounting structures. Still further, it is understood that in some embodiments the mounting structure 146 may have a different shape, size, or position (e.g., the mounting structure 146 may run the length of the substrate 102, the mounting structure 146 may be positioned at the center of the substrate 102 rather than at an edge of the substrate 102, the mounting structure 146 may be taller in z-dimension, the mounting structure 146 may be shorter in z-dimension, the mounting structure 146 may be longer in y-dimension, the mounting structure 146 may be shorter in y-dimension, etc.).

FIG. 1D is a front-view illustration (i.e., parallel to the x-z plane, as illustrated) of the bioreactor tile 100 (e.g., as illustrated and described with reference to FIGS. 1A-1C) while the bioreactor tile 100 is in use (e.g., growing plant cells and trichomes). The optical waveguide 132 of the bioreactor tile 100 has been removed from the illustration of FIG. 1D so as to prevent the illustration from being cluttered. As illustrated, the first channel 112 may supply liquid nutrient media 152 to plant cells 164 contained within a gel 154 in the second channel 114. As also illustrated, the plant cells 164 in the second channel 114 may produce trichomes 166 that extend into the third channel 116.

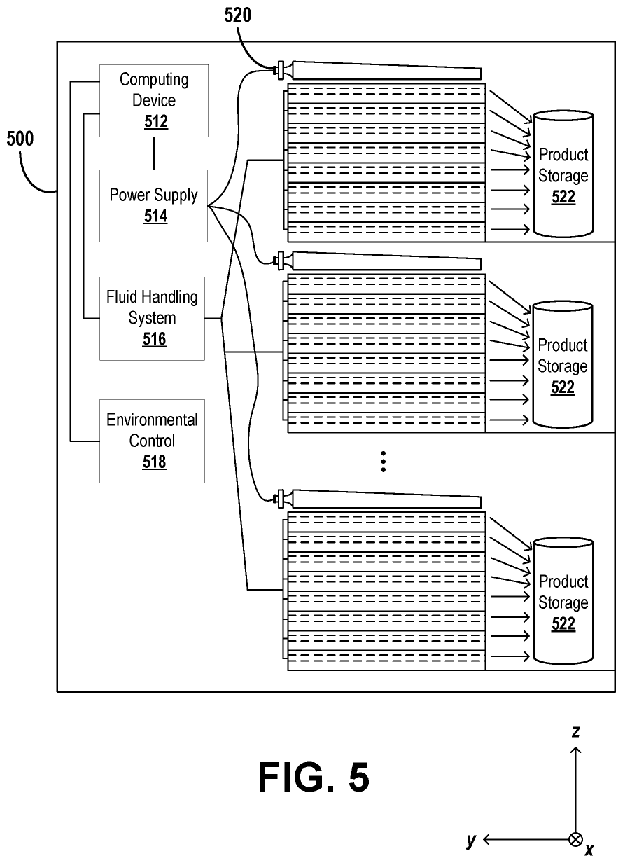

The liquid nutrient media 152 may be pumped into the bioreactor tile 100 (e.g., by a pump and/or from a tank of a bioreactor 500, as shown and described below with reference to FIG. 5). For example, the liquid nutrient media 152 may enter an inlet or an intake port (e.g., an inlet/intake port that permits fluid communication between the first channel 112 and an exterior of the bioreactor tile 100) at one end of the first channel 112. Likewise, the liquid nutrient media 152 may exit the bioreactor tile 100 (e.g., by being pumped and/or into a separate tank of a bioreactor) via an outlet or an outtake port (e.g., an outlet/outtake port that permits fluid communication between the first channel 112 and an exterior of the bioreactor tile 100). For example, the liquid nutrient media 152 may be pumped out of the bioreactor tile 100 (e.g., pumped through and out of the first channel 112) once the nutrients contained in the liquid nutrient media 152 have been depleted/used by the plant cells 164 in the second channel 114.

In some embodiments, the liquid nutrient media 152 may include an inorganic salt, a carbon source, myoinositol, glycine, a vitamin, a growth regulator, a nitrogen compound, an organic acid, a plant extract, aluminum chloride, ammonium nitrate, ammonium phosphate, ammonium hydrogen phosphate, ammonium sulfate, boric acid, calcium chloride, calcium nitrate, calcium phosphate tribasic, cobalt chloride, cupric sulfate, ferric chloride, ferric citrate, ferric ethylenediaminetetraacetic acid (EDTA), ferric sulfate, ferric tartrate, magnesium sulfate, manganese chloride, manganese sulfate, molybdenum trioxide, sodium molybdate, nickel chloride, nickel sulfate, potassium chloride, potassium iodide, potassium nitrate, potassium hydrogen phosphate, potassium sulfate, sodium EDTA, sodium molybdate, sodium nitrate, sodium phosphate, sodium hydrogen phosphate, sodium dihydrogen phosphate, sodium sulfate, zinc nitrate, zinc sulfate, activated charcoal, adenosine hemisulfate, agar, 6-benzylamino purine, alpha naphthalene acetic acid, biotin, dichloro-phenoxy acetic acid, dimethylallylaminopurine, glycine, indole-3-acetic acid, indole-3-butyric acid, isopentenyl adenine, isopentenyl adeninoside, kinetin, MES (buffer), myo-inositol, nicotinic acid, peptone, pyridoxine hydrochloride, sucrose, thiamine, thiamine hydrochloride, auxins, cytokinins, gibberellins, or plant preservatives (e.g., included in the liquid nutrient media 152 at about 1% by volume). It is understood that these are provided solely as examples and that other types of liquid nutrient media 152 are also possible and are contemplated herein. Some of these types of nutrients may be suspended and/or dissolved in a liquid (e.g., water) for easier uptake by the plant cells 164. In some embodiments, the liquid nutrient media 152 may be made into gel formulations In some embodiments, the liquid nutrient media 152 may include a calli-induction media (e.g., including a base of Gamborg's B5 Basal Media combined with 50 mg/L myo-inositol, 10 mg/L of thiamine HCl, 1 g/L of casein hydrolysate, 3% sucrose, 1 mg/L of 1-napthaleneacetic acid, and 1 mg/L benzylamino purine). Further, in some embodiments, liquid formulations may be made into gel formulations by adding gel powder prior to autoclaving. Additionally or alternatively, in some embodiments, the liquid nutrient media 152 may include trichome-induction media (e.g., including a base of Gamborg's B5 Basal Media combined with 50 mg/L myo-inositol, 10 mg/L of thiamine HCl, 1 g/L of casein hydrolysate, 3% sucrose, 1 mg/L of 1-napthaleneacetic acid, and 1 mg/L benzylamino purine, as well as 0.5-1.0 mg/L of thidiazuron and 3 mg/L gibberellic acid added after autoclaving).

Additionally or alternatively, the gel 154 may include agar gel, agarose gel, alginate gel, gelatin gel, acrylamide gel, silica gel, cellulose gel, methylcellulose gel, and/or a highly purified, natural heteropolysaccharide that forms stable, agar-like gels when exposed to soluble salts. It is understood that these are provided solely as examples and that other types of gels 154 are also possible and are contemplated herein.

Still further, the plant cells 164 may include parenchymal plant cells (e.g., originally provided in protoplast cell cultures, suspension cell cultures, or micro-calli cell cultures), in some embodiments. The parenchymal cells may include Cannabis sativa cells, Artemisia annua cells, Chrysanthemum cinerariifolium cells, Chrysanthemum coccineum cells, Gossypium hirsutum cells, Gossypium barbadense cells, Gossypium arboreum cells, Gossypium herbaceum cells, Lavandula angustifolia cells, Arabidopsis thaliana cells, Mentha x piperita cells, and/or Mentha haplocalyx cells, in various embodiments. It is understood that these are provided solely as examples and that other types of plant cells 164 are also possible and are contemplated herein (e.g., spongey mesenchymal cells or palisade cells).

The plant cells 164 may be interspersed throughout the gel 154 matrix (or cultured in a neighboring channel, based on embodiment). While only one plant cell 164 is illustrated in FIG. 1D, it is understood that this is done solely for illustration and that, in many embodiments, tens, hundreds, thousands, millions, etc. of plant cells may be present within the gel 154 of the second channel 114. In some embodiments, the plant cells 164 may have been seeded into an unset gel mixture (e.g., an unset version of the gel 154, sometimes referred to as a gel precursor media). That unset gel mixture with the seeded plant cells 164 therein may be referred to as a cellular precursor. The cellular precursor may have been infused into the second channel 114 and then allowed to solidify (e.g., over a predetermined amount of time, such as between 0.25 hours and 2.0 hours) or caused to solidify (e.g., by infusing a solidifying agent, such as CaCl₂)) to form the gel 154 with the plant cells therein 164. The gel 154 may have a low enough density so as to permit the plant cells 164 to grow, multiply, and generate the trichomes 166 that extend into the third channel 116. In some embodiments, prior to infusing the cellular precursor into the second channel 114, a sterilization procedure may be performed (e.g., to sterilize the bioreactor tile 100). The sterilization procedure may include heating the substrate 102 (with the channels 112, 114, 116 defined therein) and/or the optical waveguide 132 to a first predetermined temperature (e.g., to between 175° C. and 185° C.) for a first predetermined time period (e.g., 5 minutes, 10 minutes, 15 minutes, 30 minutes, 45 minutes, 1 hour, 2 hours, 3 hours, etc.), followed by cooling the substrate 102 (with the channels 112, 114, 116 defined therein) and/or the optical waveguide 132 to a second predetermined temperature (e.g., between 20° C. and 25° C.). Additionally or alternatively, the sterilization procedure may include a radiation procedure (e.g., illumination with ultraviolet light) and/or a chemical sterilization procedure. For example, the sterilization procedure may be a chemical sterilization procedure that includes applying a 7%-10% bleach solution, followed by autoclaving with distilled water, followed by applying a 80% ethanol solution, followed by autoclaving with distilled water, followed by drying (e.g., using clean air). Other sterilization procedures are also possible and are contemplated herein (e.g., including procedures that apply detergent, H₂O₂, plasma, dry heat, and/or steam).

In order to grow the plant cells 164, one or more sets of environmental conditions may be provided (e.g., provided to the second channel 114 or the third channel 116 of the bioreactor tile 100). Such sets of environmental conditions may be provided by a larger bioreactor (e.g., the bioreactor 500 shown and described below with reference to FIG. 5). Further providing such a set of environmental conditions may include providing a temperature of between 20° C. and 25° C. to the second channel 114 or the third channel 116, providing a periodic lighting condition (e.g., using a illumination source and/or an optical waveguide, such as the optical waveguide 132 illustrated in FIGS. 1A and 1C) that repeatedly alternates from about 16 hours of light to about 8 hours of dark, and providing a calli-induction media (e.g., as part of the liquid nutrient media 152) in the first channel 112. In some embodiments, instead of providing between 20° C. and 25° C. continuously, a temperature may be provided of between 18° C. and 30° C. during the 16 hours of light, while a temperature of between 5° C. and 10° C. is provided during the 8 hours of darkness. Still further, in some embodiments, providing such a set of environmental conditions may also include providing an air humidity of between 30% and 50% (e.g., in the air supplied to the plant cells 164 through via the third channel 116).

As illustrated in FIG. 1D, the gel 154 and plant cells 164 in the second channel 114 may be separated from the liquid nutrient media 152 in the first channel 112 by the first partial wall structure 122. Based on the first partial wall structure 122 and the surface tension in the liquid nutrient media 152 and the gel 154/plant cell 164 mixture, the two channels may be effectively prevented from mixing within one another while nutrients can still be transported from the liquid nutrient media 152 to the plant cells 164.

The gel 154 (e.g., in liquid form) and plant cell 164 mixture may be pumped into the bioreactor tile 100 (e.g., by a pump and/or from a tank of a bioreactor 500, as shown and described below with reference to FIG. 5). For example, a cellular precursor that includes the gel 154 and the plant cells 164 may enter an inlet or an intake port (e.g., an inlet/intake port that permits fluid communication between the second channel 114 and an exterior of the bioreactor tile 100) at one end of the second channel 114. Likewise, the gel 154 and plant cell 165 mixture may exit the bioreactor tile 100 (e.g., by being pumped into a separate tank of a bioreactor) via an outlet or an outtake port (e.g., an outlet/outtake port that permits fluid communication between the second channel 114 and an exterior of the bioreactor tile 100). For example, the gel 154 and plant cell 164 mixture may be pumped out of the bioreactor tile 100 (e.g., pumped through the second channel 114) once the plant cells 164 have ceased producing trichomes and/or once the trichomes 166 in the third channel 116 are harvested. In some embodiments, the gel 154 and plant cell 164 mixture may be used for multiple cycles of producing trichomes/harvesting trichomes prior to being pumped out of the bioreactor tile 100.

As also illustrated in FIG. 1D, the gel 154 and plant cells 164 in the second channel 114 may be separated from the trichomes 166 in the third channel 116 by the second partial wall structure 124. Based on the second partial wall structure 124 and the surface tension in the gel 154/plant cell 164 mixture, the two channels may be effectively prevented from mixing within one another (e.g., the gel 154 does not penetrate into the third channel 116 even when in a liquid state) while trichomes 166 produced by the plant cells 164 in the second channel 114 can still extend into the third channel 116.

The trichomes 166 may be produced by the plant cells 164. For example, Cannabis sativa parenchymal cells may produce Cannabis sativa trichomes. Further, in some embodiments, the trichomes 166 may contain products (e.g., chemicals) that are ultimately to be harvested from the bioreactor tile 100. For example, the trichomes may produce/contain cannabidiol, tetrahydrocannabinol, artemisinin, pyrethrum, camphor, glucosinolate, linalool, linalyl acetate, menthol, or peppermint camphor.

In order to cause the plant cells 164 to produce the trichomes 166, one or more sets of environmental conditions may be provided (e.g., provided to the second channel 114 or the third channel 116 of the bioreactor tile 100). Such sets of environmental conditions may be provided by a larger bioreactor (e.g., the bioreactor 500 shown and described below with reference to FIG. 5). Further providing such a set of environmental conditions may include providing a temperature of between 20° C. and 25° C. to the second channel 114 or the third channel 116, providing a periodic lighting condition (e.g., using a illumination source and/or an optical waveguide, such as the optical waveguide 132 illustrated in FIGS. 1A and 1C) that repeatedly alternates from about 12 hours of light to about 12 hours of dark, and providing a trichome-induction media (e.g., as part of the liquid nutrient media 152) in the first channel 112. In some embodiments, the trichome-induction media may include plant hormones that induce callus formation, which results in the production of trichomes by the plant cells 164.

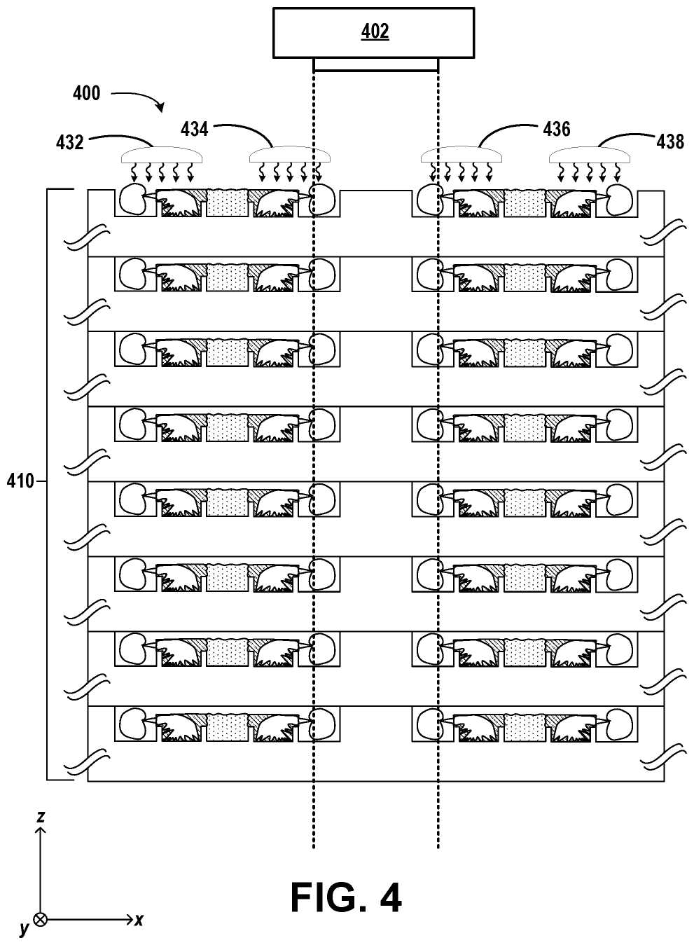

Once the trichomes 166 reach a mature state, the trichomes 166 and/or the products within the trichomes 166 may be harvested from the third channel 116. In some embodiments, one or more optical monitoring systems (e.g., including a camera 402, as shown and described with reference to FIG. 4) may be used to monitor the trichomes 166 in order to determine when the trichomes 166 have reached a mature state for harvesting. Harvesting the trichomes 166 and/or the products within the trichomes 166 may include shearing the trichomes from the underlying plant cells 164 (e.g., the underlying parenchymal cells) and extracting the trichomes from the third channel 116 by flushing the third channel 116 with a liquid flow (e.g., by infusing extractant, such as ethanol, ice-cold water, or another solvent, into the third channel 116). Thereafter, a separation step may be performed (e.g., within a different region of a bioreactor, such as the bioreactor 500 shown and described below with reference to FIG. 5) to separate the product contained within the trichomes 166 from the trichomes 166 and/or to separate the product (e.g., cannabidiol) from the extractant (e.g., by falling-film evaporation, roto-evaporation, drying, distillation, short-path distillation, and/or lyophilization). Alternatively, in some embodiments the trichomes 166 may be ruptured in order to release the product into the third channel 116, and the product may be retrieved from the third channel 116 (e.g., by flushing the third channel 116 with a liquid flow). In such embodiments, the ruptured trichomes may be sheared/extracted thereafter (e.g., separately from the products).

In some embodiments, harvesting the trichomes 166 or the products within the trichomes 166 may include providing a temperature of less than 4° C. to the second channel 114 and/or the third channel 116. Further, harvesting the trichomes 166 or the products within the trichomes 166 may include providing no illumination light (e.g., from the optical waveguide 132) to the second channel 114 or the third channel 116. Additionally, harvesting the trichomes 166 or the products within the trichomes 166 may include flowing low-temperature (e.g., between −75° C. and 0° C.) extractant through the third channel 116 at a sufficient flow rate so as to shear the trichomes 166 from the plant cells 164. In some embodiments, the extractant may include one or more abrasive components (e.g., glass beads) to assist in shearing the trichomes 166 from the plant cells 164.

In some embodiments, once the trichomes 166 and/or the products within the trichomes 166 are harvested from the third channel 116, the second channel 114 and/or the first channel 112 may also be flushed. For example, the gel 154 and the plant cells 164 within the gel 154 may be flushed from the second channel 114 (e.g., by liquefying the gel 154, such as via heating and/or enzymatic digestion, and using a fluid flow to force the liquefied gel 154 out of an outlet of the second channel 114). Additionally or alternatively, the liquid nutrient media 152 may be flushed from the first channel 112 (e.g., using a fluid flow to force the liquid nutrient media 152 out of an outlet of the first channel 112).

In other embodiments, once the trichomes 166 and/or the products within the trichomes 166 are harvested from the third channel 116, the plant cells 164 may be caused to regrow trichomes 166. For example, the liquid nutrient media 152 may be replaced with fresh calli-induction media and/or may be replaced with fresh trichome-induction media to cause the plant cells 164 to produce new trichomes 166 for harvesting. Such a process may be repeated multiple times to harvest multiple cycles of trichomes 166 without the need for replacing/regrowing the underlying plant cells 164 (e.g., assuming that the harvesting of the trichomes 166 in each cycle does not damage the underlying plant cells 164/the underlying gel 154 and/or does not compromise sterility within the bioreactor tile 100).

FIG. 2 is an isometric illustration of a bioreactor tile 200, according to example embodiments. Like the bioreactor tile 100 illustrated in FIGS. 1A-1D, the bioreactor tile 200 may include a substrate 202 having a first channel 212 (i.e., a nutrient channel 212), a second channel 214 (i.e., a plant cell channel 214), and a third channel 216 (i.e., product channel 216) defined therein. The first channel 212 may be separated from the second channel 214 by a first partial wall structure 222 and the second channel 214 may be separated from third channel 216 by a second partial wall structure 224. Also like the bioreactor tile 100 of FIGS. 1A-1D, the bioreactor tile 200 may also include an optical waveguide 232 (e.g., a SiO₂ optical waveguide). However, unlike the optical waveguide 132 of FIGS. 1A and 1C, the optical waveguide 232 of the bioreactor tile 200 in FIG. 2 is a surface-integrated optical waveguide.

Because the optical waveguide 232 illustrated in FIG. 2 is a surface-integrated optical waveguide, the optical waveguide 232 may be attached to or fabricated directly onto a surface of the substrate 202 (e.g., a surface of the substrate 202 that is adjacent to the third channel 216). For example, the optical waveguide 232 may be deposited onto the substrate 202 using a CVD process (e.g., a plasma-enhanced chemical vapor deposition process), deposited onto the substrate 202 using a sol-gel deposition process, sputtered onto the substrate 202, or grown on the substrate 202 (e.g., using a dry or wet thermal oxidation process). Also, the optical waveguide 232 may provide illumination light (e.g., illumination light received at the first end of the optical waveguide 232) to the second channel 214 or the third channel 216 (in order to provide conditions conducive to the growth of plant cells in the second channel 214 and/or the growth of trichome cells in the third channel 216) from an edge surface (e.g., as opposed to a bottom surface like the optical waveguide 132 illustrated in FIGS. 1A and 1C). This is illustrated in FIG. 2 by the light rays emitted from the edge surface of the optical waveguide 232. Similar to the bottom surface of the optical waveguide 132 in FIGS. 1A and 1C, providing the illumination light via the edge surface to the second channel 214 and/or the third channel 216 may occur based on one or more surface features present on the edge surface of the optical waveguide 232 that allow illumination light to escape the optical waveguide 232 (e.g., that interrupt total internal reflection) as the illumination light propagates in the optical waveguide 232. For example, the edge surface of the optical waveguide 232 may include diffractive features, longitudinal striations (e.g., running parallel to the y-axis illustrated in FIG. 2), lateral striations (e.g., running parallel to the z-axis illustrated in FIG. 2), or pits.

Also like the optical waveguide 132 illustrated in FIGS. 1A and 1C, one or more surfaces of the optical waveguide 232 may be tapered. For example, a top surface of the optical waveguide 232 (e.g., a surface of the optical waveguide 232 having the highest z-position) may be tapered (e.g., from a first end of the optical waveguide 232 to a second end of the optical waveguide 232). Such a tapering may provide for a relatively uniform intensity of illumination light provided to the second channel 214 and/or the third channel 216 by the optical waveguide 232. In other embodiments, other shapes of optical waveguide 232 may be used. For example, the intensity of the illumination light provided to the second channel 214 and/or the third channel 216 may substantially vary along the length (e.g., y-position) of the second channel 214 and/or the third channel 216.

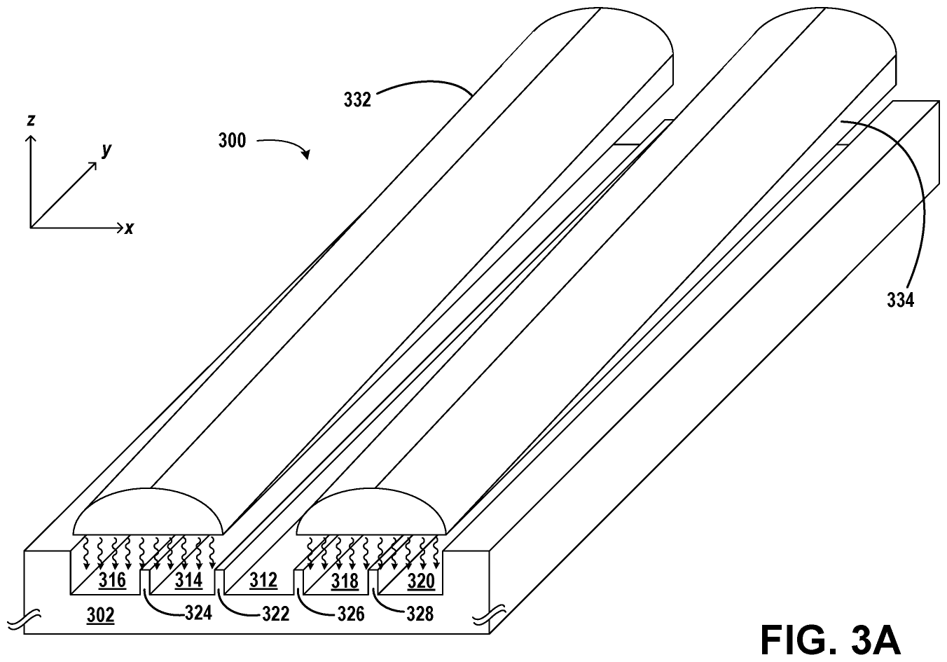

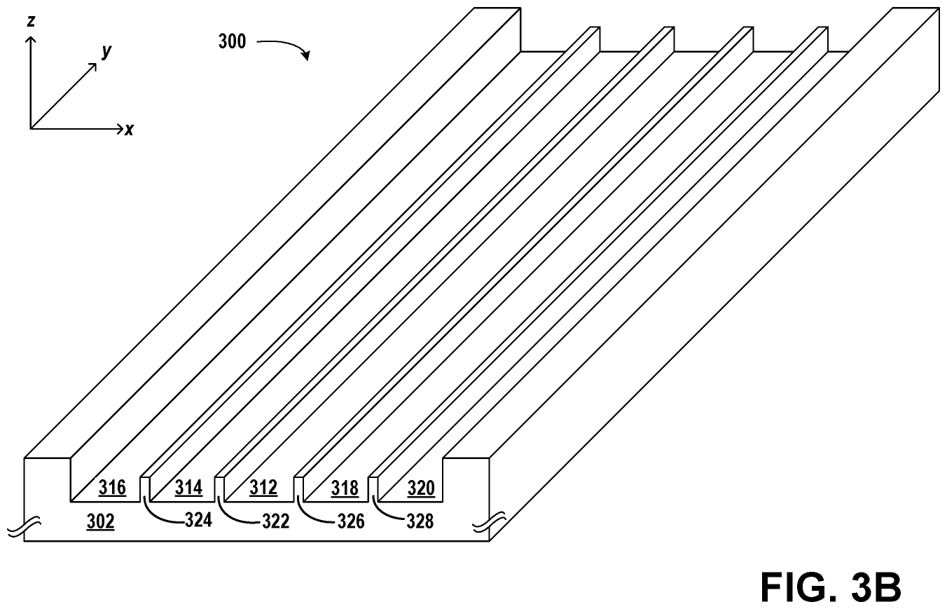

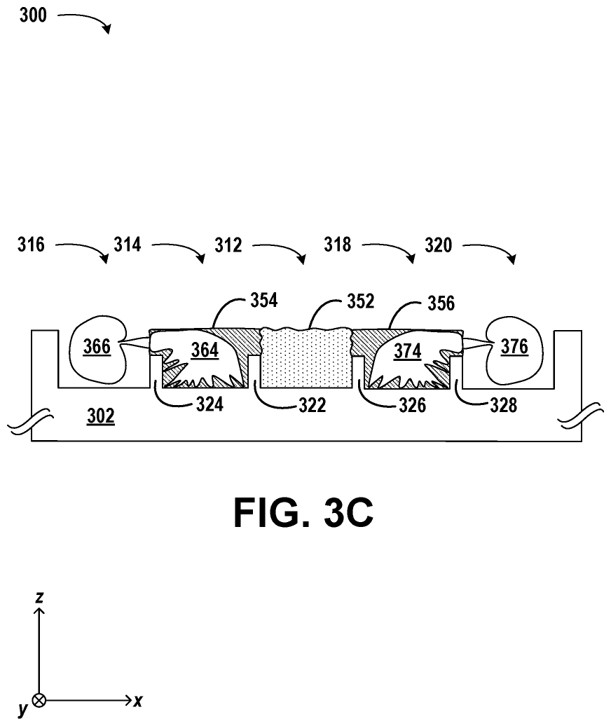

FIG. 3A is an isometric illustration of a bioreactor tile 300, according to example embodiments. The bioreactor tile 300 may include a substrate 302 having a first channel 312 (i.e., nutrient channel 312), a second channel 314 (i.e., plant cell channel 314), a third channel 316 (i.e., product channel 316), a fourth channel 318 (i.e., plant cell channel 318), and a fifth channel 320 (i.e., product channel 320) defined therein. The first channel 312 may be separated from the second channel 314 by a first partial wall structure 322, the second channel 314 may be separated from third channel 316 by a second partial wall structure 324, the first channel 312 may be separated from the fourth channel 318 by a third partial wall structure 326, and the fourth channel 318 may be separated from the fifth channel 320 by a fourth partial wall structure 328. The bioreactor tile 300 may also include a first optical waveguide 332 and a second optical waveguide 334. The arrangement of the bioreactor tile 300 of FIG. 3A may be referred to as a double-sided arrangement, and may be similar to the single-sided arrangement illustrated in FIGS. 1A-1D (e.g., with the exception that the double-sided arrangement may permit additional production of product per unit time, per unit area on the substrate 302, and/or per unit area within the bioreactor tile 300, when compared to the substrate 102/bioreactor tile 100 of the single-sided arrangement of FIGS. 1A-1D).

FIG. 3B is an isometric illustration of the same bioreactor tile 300 as FIG. 3A, with the optical waveguides 332, 334 removed to make the underlying structures of the first channel 312, the second channel 314, the third channel 316, the fourth channel 318, the fifth channel 320, the first partial wall structure 322, the second partial wall structure 324, the third partial wall structure 326, and the fourth partial wall structure 328 more clearly visible. As illustrated in FIG. 3B, the first channel 312, the second channel 314, the third channel 316, the fourth channel 318, and the fifth channel 320 may run substantially parallel to one another (e.g., parallel to the y-axis). In some embodiments, the first channel 312, the second channel 314, the third channel 316, the fourth channel 318, and/or the fifth channel 320 may be fluidic channels (e.g., microfluidic channels or larger-scale channels).

The first optical waveguide 332 may be similar to the optical waveguide 132 illustrated and described with reference to FIGS. 1A and 1C. For example, the first optical waveguide 332 may be configured to provided illumination light to the second channel 314 and/or the third channel 316, may have surface features that allow illumination light to escape a bottom surface of the first optical waveguide 332, and/or may be tapered from a first end to a second end of the first optical waveguide 332. Likewise, the second optical waveguide 334 may be similar to the optical waveguide 132 illustrated and described with reference to FIGS. 1A and 1C. For example, the second optical waveguide 334 may be configured to provided illumination light to the fourth channel 318 and/or the fifth channel 320, may have surface features that allow illumination light to escape a bottom surface of the second optical waveguide 334, and/or may be tapered from a first end to a second end of the second optical waveguide 334. It is understood that the arrangement of FIG. 3A is provided solely as an example and that other embodiments are also possible. For example, in some embodiments, each separate channel may have its own respective optical waveguide (e.g., five optical waveguides, one for each of the five channels 312, 314, 316, 318, 320), each of the plant cell channels 314, 318 and the product channels 316, 320 may have its own respective optical waveguide (e.g., four optical waveguides, one for each of the first channel 312, the second channel 314, the fourth channel 318, and the fifth channel 320), or a single waveguide may be used to provide illumination light to all the channels 312, 314, 316, 318, 320. Other embodiments are also possible and are contemplated herein (e.g., embodiments having multiple surface-integrated optical waveguides, similar to the surface-integrated optical waveguide illustrated in FIG. 2).

FIG. 3C is a front-view illustration (i.e., parallel to the x-z plane, as illustrated) of the bioreactor tile 300 illustrated and described with reference to FIGS. 3A and 3B while the bioreactor tile 300 is in use (e.g., growing plant cells and trichomes). The optical waveguides 332, 334 of the bioreactor tile 300 have been removed from the illustration of FIG. 3C so as to prevent clutter. As illustrated, the first channel 312 may supply liquid nutrient media 352 to plant cells 364 contained within a gel 354 in the second channel 314 and to plant cells 374 contained within a gel 356 in the fourth channel 318. As also illustrated, the plant cells 364 in the second channel 314 may produce trichomes 366 that extend into the third channel 316 and the plant cells 374 in the fourth channel 318 may produce trichomes 376 that extend into the fifth channel 320. The substances contained within and the actions performed using the first channel 312 may be similar to that of the first channel 112 as shown and described with reference to FIG. 1D. Similarly, the substances contained within and the actions performed using the second channel 314 and the fourth channel 318 may be similar to that of the second channel 114 as shown and described with reference to FIG. 1D. Additionally, the substances contained within and the actions performed using the third channel 316 and the fifth channel 320 may be similar to that of the third channel 116 as shown and described with reference to FIG. 1D.

FIG. 4 is a front-view illustration (i.e., parallel to the x-z plane, as illustrated) of a bioreactor tile 400. The bioreactor tile 400 may include a plurality of layers 410 (e.g., each layer including a substrate with multiple channels defined therein, similar to the substrate 102 of FIGS. 1A-1D, the substrate 202 of FIG. 2, or the substrate 302 of FIGS. 3A-3C). As such, the bioreactor tile 400 may be referred to as a multi-layer bioreactor tile. Further, the bioreactor tile 400 may include a plurality of optical waveguides (e.g., a first optical waveguide 432, a second optical waveguide 434, a third optical waveguide 436, and a fourth optical waveguide 438). The optical waveguides 432, 434, 436, 438 may direct illumination light to the channels positioned underneath the respective optical waveguides 432, 434, 436, 438. For example, the plant cells, trichomes, gel, liquid nutrient media, and substrate in the various layers 410 may be transparent or substantially translucent, thereby allowing illumination light from the optical waveguides 432, 434, 436, 438 to be provided vertically (e.g., along the z-direction) to all layers 410. Though not labeled with reference numerals (to avoid unnecessary clutter of the drawing), the respective channels in each of the plurality of layers 410 may be providing nutrients, growing plant cells, or housing produced trichomes, as illustrated. Also illustrated in FIG. 4 (though not necessarily a component of the bioreactor tile 400, itself) is a camera 402 positioned above at least some subset of the channels in the plurality of layers 410.

Each layer within the plurality of layers 410 in the bioreactor tile 400 may be used to produce product (e.g., contained within the trichomes produced in that respective layer). As such, the bioreactor tile 400 may have higher throughput (e.g., production of product per unit volume or per unit time) than the bioreactor tiles 100, 200, 300 illustrated in FIGS. 1A-3C. In some embodiments, each of the layers 410 may be growing the same types of plant cells and producing the same products (e.g., contained within trichomes). However, this need not be the case. In some embodiments, different layers could be growing different plant cells and/or producing different products from one another. Further, in some embodiments, each of the channels that contain the same substances (e.g., within the same layer and/or across multiple layers) may be supplied/controlled together. For example, each of the liquid nutrient media channels in all layers of the bioreactor tile 400 may receive liquid nutrient media from inlets of the respective channels, and each of the inlets of the respective channels may be fed by the same supply (e.g., tank containing liquid nutrient media) and/or connected to the same pump (e.g., within a bioreactor, such as the bioreactor 500 shown and described below within reference to FIG. 5). Similarly, outlets of the same types of channels may also be connected. For example, outlets of the product channels may provide harvested trichomes to the same product storage (e.g., product tank). Other examples of the various layers being tied to one another are also possible and are contemplated herein. For example, in some embodiments, a bioreactor tile may include one or more separate dilution channels (e.g., one dilution channel per layer, multiple dilution channels per layer, or a single dilution channel for the entire bioreactor tile) that is usable to evaluate the content of a mixture in the tile (e.g., a chemical composition of a liquid nutrient media that is flowed through the dilution channel(s)). It is also understood that in some embodiments (e.g., embodiments where the plant cells being grown and/or the product being produced in the different layers are different) the layers may be operated independently.

The camera 402 may be a component of the bioreactor tile 400 or of a larger bioreactor (e.g., the bioreactor 500 illustrated and described below with reference to FIG. 5), in various embodiments. As illustrated in FIG. 4, the camera 402 may be positioned above the plurality of layers 410. For example, the camera 402 may be positioned above the product channels in an adjacent double-sided arrangements of channels (e.g., based on the product channels being defined within the substrates such that there is sufficient spacing between the product channels in the adjacent double-sided arrangements so as to permit imaging of the trichomes within the product channels). As such, the camera 402 may be configured to capture images (e.g., red-green-blue (RGB) images) of trichomes within the product channels (e.g., both of the trichomes in the product channels in the top bioreactor tile layer and trichomes in lower bioreactor tile layers). In some embodiments, the camera 402 may include one or more lenses or mirrors (e.g., may be integrated within an optical microscope) to capture images at the appropriate scale. Using images captured by the camera 402 (e.g., multiple images captured over time), the maturity state of the trichomes may be monitored. In some embodiments, there may be additional cameras (e.g., placed above additional product channels). Further, in some embodiments, the camera 402 (or additional cameras) may be positioned and configured to capture images of other channels (e.g., the plant cell channels to monitor the growth of the plant cells). While the camera 402 described herein may be an optical camera used to capture RGB images, it is understood that other types of imaging devices (e.g., to monitor the growth of the plant cells and/or trichomes) using other imaging modalities are also possible and are contemplated herein. For example, in some embodiments, the camera 402 may be replaced by or augmented by one or more devices capable of performing hyperspectral imaging, Raman spectroscopy, and/or fluorescence spectroscopy. Other imaging modalities are also possible and are contemplated herein.

FIG. 5 is a schematic illustration of a bioreactor 500, according to example embodiments. The bioreactor 500 may include a cabinet that includes a plurality of bioreactor tiles (e.g., the bioreactor tile 100 illustrated in FIGS. 1A-1D, the bioreactor tile 200 illustrated in FIG. 2, the bioreactor tile 300 illustrated in FIGS. 3A-3C, or the bioreactor tile 400 illustrated in FIG. 4), a computing device 512, a power supply 514, a fluid handling system 516, an environmental control 518, a plurality of illumination sources 520, and product storage 522 (e.g., a product storage 522 associated with each bioreactor tile). For example, there may be a plurality of multi-layer bioreactor tiles (e.g., a plurality of copies of the bioreactor tile 400 illustrated in FIG. 4) that are arranged on various shelves of the bioreactor 500 (e.g., each shelf being individually retractable/removable/swappable in order to swap out bioreactor tiles). As illustrated by the three vertical dots in FIG. 5, while only three bioreactor tiles/three product storage 522 are illustrated, it is understood that additional bioreactor tiles/product storage 522 may also be present within the bioreactor 500 (e.g., four bioreactor tiles, five bioreactor tiles, seven bioreactor tiles, eight bioreactor tiles, nine bioreactor tiles, ten bioreactor tiles, sixteen bioreactor tiles, twenty bioreactor tiles, thirty bioreactor tiles, thirty six bioreactor tiles, forty bioreactor tiles, fifty bioreactor tiles, sixty bioreactor tiles, sixty four bioreactor tiles, seventy bioreactor tiles, eighty bioreactor tiles, ninety bioreactor tiles, one hundred bioreactor tiles, one hundred and ten bioreactor tiles, one hundred and twenty bioreactor tiles, one hundred and twenty eight bioreactor tiles, etc.).

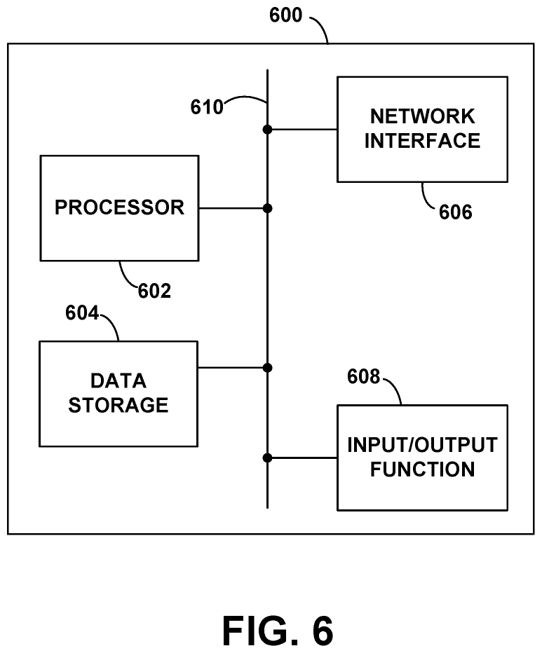

The computing device 512 may serve as a controller for one or more components of the bioreactor 500. For example, as illustrated by the lines connected to the computing device 512 (e.g., representing one or more communicative connections, such as wireline connections or wireless connections), the computing device 512 may receive (and possibly store) information from and/or provide instructions to the power supply 514, the fluid handling system 516, and/or the environmental control 518. In some embodiments, the computing device 512 may also be connected to one or more sensors within one or more product storage 522 (e.g., to monitor an amount of product contained in the one or more product storage 522). In order to perform such tasks, the computing device 512 may execute (e.g., using a processor) instructions stored in a memory (e.g., a non-transitory computer-readable medium). An example computing device 600 is described below with reference to FIG. 6. The computing device 600 may be used as or a component of the computing device 512 illustrated in FIG. 5, in some embodiments. Further, while the computing device 512 is shown as integrated into the bioreactor 500 in FIG. 5, it is understood that other embodiments are also possible and are contemplated herein (e.g., the computing device 512 may be a remote computing device, such as a cloud computing device, that communicates with one or more components of the bioreactor 500 from a remote location, such as over the public Internet, over WiFi, or using BLUETOOTH®).

In some embodiments, the computing device 512 may cause the power supply 514 to provide power to one or more (e.g., a selected subset) of the illumination sources 520 such that the illumination sources 520 provide illumination light to the plant cells and/or trichomes in the bioreactor tiles (e.g., via optical waveguides of the bioreactor tiles). This may be triggered based on imaging (e.g., optical monitoring using a camera connected to the computing device 512, such as the camera 402 illustrated in FIG. 4) of the trichomes and/or the plant cells. For example, at a given maturity level of the plant cells, the computing device 512 may cause the power supply 514 to provide power to one or more of the illumination sources 520 in 12 hour increments (e.g., a periodic lighting condition of 12 hours of light followed by 12 hours of darkness).

The power supply 514 may be connected to the computing device 512 (e.g., to receive instructions from the computing device 512) and/or to the illumination sources 520 (e.g., as illustrated by the wired connections in FIG. 5). In some embodiments, the power supply may include a capacitor, a battery, a connection to an external power supply (e.g., via a wall outlet), a generator, a converter/rectifier (e.g., to convert from an alternating current (AC) to a direct current (DC) power supply), etc. The power supply 514 may also include one or more switches (e.g., transistors) capable of selecting a subset of illumination sources 520 within the bioreactor 500 to which to apply power. In this way, the power supply 514 can cause subsets of the illumination sources 520 to selectively provide illumination light to the respective bioreactor tiles.

The fluid handling system 516 may include one or more fluid supplies (e.g., tanks including fluid mixtures and/or tanks including single fluid components which can be mixed by the fluid handling system 516 to generated fluid mixtures). Such fluid supplies may be connected to the channels of the bioreactor tiles (e.g., to inlets of the channels of the bioreactor tiles), which is illustrated in FIG. 5 by the lines connecting the fluid handling system 516 to the channels of the bioreactor tiles. The fluid supplies may be connected to the channels of the bioreactor tiles via tubing, pipes, or other fluid lines. In some embodiments, the fluid handling system 516 may also include one or more valves (e.g., solenoids or pneumatic valves) usable to open and/or close the respective fluidic connections between the fluid supplies and the bioreactor tiles. In this way, the fluid handling system 516 can select a subset of the bioreactor tiles (or even, in some embodiments, a subset of the layers across all bioreactor tiles or, even further, specific channels across all bioreactor tiles) to receive certain fluids (e.g., liquid nutrient media, extractant, cellular precursor, etc.). Still further, in some embodiments, the fluid handling system 516 may include one or more pumps (e.g., plunger pumps, diaphragm pumps, piston pumps, hydraulic pumps, peristaltic pumps, rotary vane pumps, screw pumps, gear pumps, etc.) used to cause fluid to flow from the fluid supplies to the bioreactor tiles and/or to force fluid out of the bioreactor tiles into the product storage 522. Additionally or alternatively, in some embodiments, one or more components of the fluid handling system 516 may include one or more mass flow controllers (e.g., to measure how much fluid has passed through a certain component of the fluid handling system 516). Measurements from the one or more mass flow controllers may be communicated to the computing device 512 in order for the computing device 512 to make control determinations, for example.

The environmental control 518 may include one or more heating or cooling devices. Such heating or cooling devices may be controlled by the computing device 512, for example, in order to maintain appropriate environmental conditions within the bioreactor 500 (e.g., appropriate environmental conditions to grow plant cells and/or cause the plant cells to produce trichomes). Additionally or alternatively, in some embodiments, the environmental control 518 may include a temperature sensor (e.g., a thermometer) configured to measure a current temperature within the bioreactor 500, as a whole, or within one specific region of the bioreactor 500 (e.g., within one of the bioreactor tiles). The temperature sensor may provide temperature measurements to the computing device 512 so the computing device 512 can make control decisions for the bioreactor 500 based on the given temperature within the bioreactor 500/within a given region of the bioreactor 500. Still further, in some embodiments, the environmental control 518 may include one or more humidifiers (e.g., configured to be controlled by the computing device 512 so as to maintain the humidity around one or more of the bioreactor tiles or around specific channels within one or more of the bioreactor tiles).

The illumination sources 520 may provide illumination light to the optical waveguides of the bioreactor tiles in the bioreactor 500 (e.g., when supplied with power from the power supply 514). In some embodiments, the illumination sources 520 may include one or more LEDs, light bulbs, lasers, etc. Wavelengths for the illumination light produced by the illumination sources 520 may be within a range usable for photosynthesis within plant cells of in the bioreactor tiles, for example. Additionally, in some embodiments, the illumination sources 520 may produce a wide range of wavelengths, from which one or more optical filters selects a narrower range of wavelengths to provide to the optical waveguides in the bioreactor tiles. Further, in some embodiments, the illumination sources 520 may be located far enough from the bioreactor tiles within the bioreactor 500 so as not to cause heating that adversely affects the environmental conditions within the channels of the bioreactor tiles. In some embodiments, illumination light from the illumination sources 520 may be coupled to the optical waveguides of the bioreactor tiles using free-space coupling. However, it is understood that other optical coupling methods are also possible and are contemplated herein. For example, one or more Bragg gratings, one or more lenses, one or more mirrors, one or more optical fibers, etc. may be used to couple the illumination light from the illumination sources 520 into the respective optical waveguides.

The product storage 522 may include one or more tanks configured to store the product generated by the bioreactor tiles (e.g., generated by trichomes in the bioreactor tiles and extracted from the bioreactor tiles upon the trichomes reaching maturity) and/or used liquid nutrient media/plant cell and gel mixtures from the bioreactor tiles. In some embodiments, one or more fluidic connections (e.g., pipes, tubes, fluid lines, etc.) may connect one or more of the channels in the respective bioreactor tiles to the product storage 522. While a product storage 522 is illustrated in FIG. 5 as corresponding to each bioreactor tile in the bioreactor 500, it is understood that each product storage 522 may represent a series of tanks/containers (e.g., one tank corresponding to product from the respective bioreactor tile and one tank corresponding to used liquid nutrient media). It is also understood that, in other embodiments, other arrangements are also possible. For example, a single product storage may be shared by multiple bioreactor tiles (e.g., all bioreactor tiles within the bioreactor 500).

FIG. 6 is a simplified block diagram exemplifying a computing device 600 (e.g., the computing device 512 illustrated and described above with respect to FIG. 5), illustrating some of the functional components that could be included in a computing device arranged to operate in accordance with the embodiments herein. Example computing device 600 could be a client device, a server device, or some other type of computational platform.

In this example, computing device 600 includes a processor 602, a data storage 604, a network interface 606, and an input/output function 608, all of which may be coupled by a system bus 610 or a similar mechanism. Processor 602 can include one or more CPUs, such as one or more general purpose processors and/or one or more dedicated processors (e.g., application specific integrated circuits (ASICs), digital signal processors (DSPs), network processors, etc.).

Data storage 604, in turn, may include volatile and/or non-volatile data storage devices and can be integrated in whole or in part with processor 602. Data storage 604 can hold program instructions, executable by processor 602, and data that may be manipulated by such program instructions to carry out the various methods, processes, or operations described herein. Alternatively, these methods, processes, or operations can be defined by hardware, firmware, and/or any combination of hardware, firmware, and software. By way of example, the data in data storage 604 may contain program instructions, perhaps stored on a non-transitory, computer-readable medium, executable by processor 602 to carry out any of the methods, processes, or operations disclosed in this specification or the accompanying drawings. The data storage 604 may include non-volatile memory (e.g., a read-only memory, ROM) and/or volatile memory (e.g., random-access memory, RAM), in various embodiments. For example, the data storage 604 may include a hard drive (e.g., hard disk), flash memory, a solid-state drive (SSD), electrically erasable programmable read-only memory (EEPROM), dynamic random-access memory (DRAM), and/or static random-access memory (SRAM). It will be understood that other types of transitory or non-transitory data storage devices are possible and contemplated within the scope of the present disclosure.

Network interface 606 may take the form of a wireline connection, such as an Ethernet, Token Ring, or T-carrier connection. Network interface 606 may also take the form of a wireless connection, such as IEEE 802.11 (WiFi), BLUETOOTH®, BLUETOOTH LOW ENERGY (BLE)®, or a wide-area wireless connection. However, other forms of physical layer connections and other types of standard or proprietary communication protocols may be used over network interface 606. Furthermore, network interface 206 may include multiple physical interfaces.

Input/output function 608 may facilitate user interaction with example computing device 600. Input/output function 608 may comprise multiple types of input devices, such as a keyboard, a mouse, a touch screen, and so on. Similarly, input/output function 608 may include multiple types of output devices, such as a screen, monitor, printer, or one or more light emitting diodes (LEDs). Additionally or alternatively, example computing device 600 may support remote access from another device, via network interface 606 or via another interface (not shown), such as a universal serial bus (USB) or high-definition multimedia interface (HDMI) port.

In some embodiments, one or more computing devices may be deployed in a networked architecture. The exact physical location, connectivity, and configuration of the computing devices may be unknown and/or unimportant to client devices. Accordingly, the computing devices may be referred to as “cloud-based” devices that may be housed at various remote locations.

### III. Example Processes

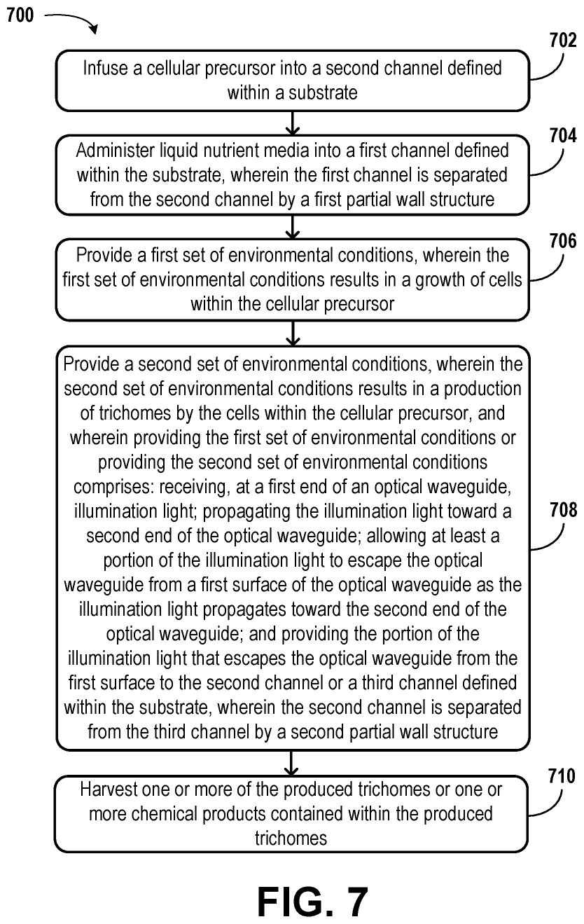

FIG. 7 is a flowchart diagram of a method 700, according to example embodiments. The method 700 may be used to grow and harvest trichome cells (e.g., using the bioreactor tile 100 illustrated in FIGS. 1A-1D, the bioreactor tile 200 illustrated in FIG. 2, the bioreactor tile 300 illustrated in FIGS. 3A-3C, the bioreactor tile 400 illustrated in FIG. 4, or the bioreactor 500 illustrated in FIG. 5).

At block 702, the method 700 may include infusing a cellular precursor into a second channel defined within a substrate.

At block 704, the method 700 may include administering liquid nutrient media into a first channel defined within the substrate. The first channel may be separated from the second channel by a first partial wall structure.

At block 706, the method 700 may include providing a first set of environmental conditions. The first set of environmental conditions may result in a growth of cells within the cellular precursor.

At block 708, the method 700 may include providing a second set of environmental conditions. The second set of environmental conditions may result in a production of trichomes by the cells within the cellular precursor. Providing the first set of environmental conditions or providing the second set of environmental conditions may include receiving, at a first end of an optical waveguide, illumination light; propagating the illumination light toward a second end of the optical waveguide; allowing at least a portion of the illumination light to escape the optical waveguide from a first surface of the optical waveguide as the illumination light propagates toward the second end of the optical waveguide; and providing the portion of the illumination light that escapes the optical waveguide from the first surface to the second channel or a third channel defined within the substrate. The second channel may be separated from the third channel by a second partial wall structure.

At block 710, the method 700 may include harvesting one or more of the produced trichomes or one or more chemical products contained within the produced trichomes.

In some embodiments, the method 700 may include performing a sterilization procedure. The sterilization procedure may include heating the substrate or the optical waveguide to a first predetermined temperature for a first predetermined time period. The sterilization procedure may also include cooling the substrate or the optical waveguide to a second predetermined temperature. In some embodiments, the first predetermined temperature may be between 175° C. and 185° C. Further, the second predetermined temperature may be between 20° C. and 25° C.

In some embodiments, the method 700 may include monitoring the growth of cells within the cellular precursor or monitoring the production of trichomes by the cells within the cellular precursor using one or more imaging modalities. The one or more imaging modalities may include capturing one or more red-green-blue (RGB) images using an optical microscope, performing hyperspectral imaging, performing Raman spectroscopy, or performing fluorescence spectroscopy.

In some embodiments of the method 700, the cellular precursor may include a gel precursor media. A gel of the gel precursor media may include agar gel, agarose gel, alginate gel, gelatin gel, acrylamide gel, silica gel, or cellulose gel. The cells within the cellular precursor may include protoplast cell cultures, suspension cell cultures, or micro-calli cell cultures. Further, in some embodiments, infusing the cellular precursor may include waiting a predetermined amount of time for the gel of the gel precursor media to solidify. For example, the predetermined amount of time may be between 0.25 hours and 2.0 hours.

In some embodiments of the method 700, providing the first set of environmental conditions may include providing a temperature of between 20° C. and 25° C. to the second channel or the third channel. Providing the first set of environmental conditions may also include providing the portion of the illumination light that escapes the optical waveguide from the first surface to the second channel or third channel for about 16 hours. Further, providing the first set of environmental conditions may include providing no illumination light to the second channel or third channel for about 8 hours (e.g., providing the first set of environmental conditions may include a periodic administration of illumination light that includes cycles of about 16 hours of illumination light followed by about 8 hours of darkness). Additionally, providing the first set of environmental conditions may include providing a calli-induction media into the first channel.

In some embodiments of the method 700, providing the second set of environmental conditions may include providing a temperature of between 20° C. and 25° C. to the second channel or the third channel. Providing the second set of environmental conditions may also include providing the portion of the illumination light that escapes the optical waveguide from the first surface to the second channel or third channel for about 12 hours. Further, providing the second set of environmental conditions may include providing no illumination light to the second channel or third channel for about 12 hours (e.g., providing the first set of environmental conditions may include a periodic administration of illumination light that includes cycles of about 12 hours of illumination light followed by about 12 hours of darkness). Additionally, providing the second set of environmental conditions may include providing a trichome-induction media into the first channel.

In some embodiments of the method 700, harvesting the one or more trichomes or the one or more chemical products contained within the produced trichomes may include providing a temperature of less than 4° C. to the second channel or the third channel. Harvesting the one or more trichomes or the one or more chemical products contained within the produced trichomes may also include providing no illumination light to the second channel or third channel. Further, harvesting the one or more trichomes or the one or more chemical products contained within the produced trichomes may include flowing low-temperature extractant through the third channel at a sufficient flow rate so as to shear the one or more trichomes from the cells within the cellular precursor. In some embodiments, the low-temperature extractant may include ethanol at a temperature of between −75° C. and 0° C.

### IV. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

The computer-readable medium can also include non-transitory computer-readable media such as computer-readable media that store data for short periods of time like register memory and processor cache. The computer-readable media can further include non-transitory computer-readable media that store program code and/or data for longer periods of time. Thus, the computer-readable media may include secondary or persistent long term storage, like ROM, optical or magnetic disks, solid state drives, compact-disc read-only memory (CD-ROM), for example. The computer-readable media can also be any other volatile or non-volatile storage systems. A computer-readable medium can be considered a computer-readable storage medium, for example, or a tangible storage device.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

Embodiments of the present disclosure may thus relate to one of the enumerated example embodiments (EEEs) listed below.

EEE 1 is a system comprising:

a substrate having a first channel, a second channel, and a third channel defined therein, wherein the first channel is separated from the second channel by a first partial wall structure, and wherein the second channel is separated from the third channel by a second partial wall structure; and

an optical waveguide configured to:
receive illumination light at a first end of the optical waveguide;
propagate the illumination light toward a second end of the optical waveguide;
allow at least a portion of the illumination light to escape the optical waveguide from a first surface of the optical waveguide as the illumination light propagates toward the second end of the optical waveguide; and
provide the portion of the illumination light that escapes the optical waveguide from the first surface to the second channel or the third channel.

EEE 2 is the system of EEE 1,

wherein the second channel is figured to house parenchymal cells suspended in gel,

wherein the first channel is configured to supply liquid nutrient media to the parenchymal cells held in the second channel, and

wherein the third channel is configured to house trichomes produced by the parenchymal cells.

EEE 3 is the system of EEE 2,

wherein the first channel comprises a first inlet and a first outlet,

wherein the first inlet and the first outlet permit fluid communication between the first channel and an exterior of the system,

wherein the second channel comprises a second inlet and a second outlet,

wherein the second inlet and the second outlet permit fluid communication between the second channel and the exterior of the system,

wherein the third channel comprises a third inlet and a third outlet, and

wherein the third inlet and the third outlet permit fluid communication between the third channel and the exterior of the system.

EEE 4 is the system of either EEE 2 or EEE 3, wherein the parenchymal cells comprise Cannabis sativa cells, Artemisia annua cells, Chrysanthemum cinerariifolium cells, Chrysanthemum coccineum cells, Gossypium hirsutum cells, Gossypium barbadense cells, Gossypium arboreum cells, Gossypium herbaceum cells, Lavandula angustifolia cells, Arabidopsis thaliana cells, Mentha x piperita cells, or Mentha haplocalyx cells.

EEE 5 is the system of any of EEEs 2-4, wherein the trichomes comprise cannabidiol, tetrahydrocannabinol, artemisinin, pyrethrum, camphor, glucosinolate, linalool, linalyl acetate, menthol, or peppermint camphor.

EEE 6 is the system of any of EEEs 2-5, wherein the liquid nutrient media comprise an inorganic salt, a carbon source, myoinositol, glycine, a vitamin, a growth regulator, a nitrogen compound, an organic acid, or a plant extract.

EEE 7 is the system of any of EEEs 2-6, wherein the third channel is configured to be infused with an extractant in order to harvest the trichomes produced by the parenchymal cells.

EEE 8 is the system of any of EEEs 1-7, wherein the substrate has a fourth channel and a fifth channel defined there, wherein the first channel is separated from the fourth channel by a third partial wall structure, wherein the fourth channel is separated from the fifth channel by a fourth partial wall structure, and wherein the system further comprises an additional optical waveguide configured to:

receive additional illumination light at a first end of the additional optical waveguide;

propagate the additional illumination light toward a second end of the additional optical waveguide;

allow at least a portion of the additional illumination light to escape the additional optical waveguide from a first surface of the additional optical waveguide as the additional illumination light propagates toward the second end of the additional optical waveguide; and

provide the portion of the additional illumination light that escapes the additional optical waveguide from the first surface of the additional optical waveguide to the fourth channel or the fifth channel.

EEE 9 is the system of any of EEEs 1-8, further comprising:

a second substrate having a fourth channel, a fifth channel, and a sixth channel defined therein,

wherein the fourth channel is separated from the fifth channel by a third partial wall structure, and wherein the fifth channel is separated from the sixth channel by a fourth partial wall structure, and

wherein the optical waveguide is further configured to provide the portion of the illumination light that escapes the optical waveguide from the first surface of the optical waveguide to the fifth channel or the sixth channel.

EEE 10 is the system of EEE 9, wherein the second substrate is positioned vertically adjacent to the substrate.

EEE 11 is the system of any of EEEs 1-10,

wherein the substrate has a fourth channel, a fifth channel, and a sixth channel defined therein,

wherein the fourth channel is separated from the fifth channel by a third partial wall structure,

wherein the fifth channel is separated from the sixth channel by a fourth partial wall structure,

wherein the fourth channel, the fifth channel, and the sixth channel are substantially parallel to the first channel, the second channel, and the third channel, respectively, and

wherein the system further comprises an additional optical waveguide configured to:
receive illumination light at a first end of the additional optical waveguide;
propagate the illumination light toward a second end of the additional optical waveguide;
allow at least a portion of the illumination light to escape the additional optical waveguide from a first surface of the additional optical waveguide as the illumination light propagates toward the second end of the additional optical waveguide; and
provide the portion of the illumination light that escapes the additional optical waveguide from the first surface of the additional optical waveguide to the fifth channel or the sixth channel.

EEE 12 is the system of EEE 11, wherein the third channel and the sixth channel are defined within the substrate adjacently to one another with a spacing therebetween, and wherein the spacing is sufficient to permit imaging of one or more substances contained within the third channel or the sixth channel.

EEE 13 is the system of any of EEEs 1-12, wherein the optical waveguide is suspended above the second channel or the third channel.

EEE 14 is the system of any of EEEs 1-12, wherein the optical waveguide is positioned on the substrate adjacent to the third channel or overlays the second channel or the third channel.

EEE 15 is the system of any of EEEs 1-14, wherein the optical waveguide is configured such that the portion of the illumination light that escapes the optical waveguide from the first surface and is provided to the second channel or the third channel is of substantially uniform intensity along a length direction of the second channel or a length direction of the third channel.

EEE 16 is the system of any of EEEs 1-14, wherein the optical waveguide is configured such that the portion of the illumination light that escapes the optical waveguide from the first surface and is provided to the second channel or the third channel has an intensity that varies along a length direction of the second channel or a length direction of the third channel.

EEE 17 is the system of any of EEEs 1-16, wherein the optical waveguide is tapered in at least one dimension between the first end and the second end.

EEE 18 is the system of any of EEEs 1-17, wherein the optical waveguide comprises:

a mixing region configured to homogenize modes or wavelengths present within the illumination light received at the first end of the optical waveguide; and

a coupling region configured to couple the homogenized illumination light from the mixing region into a main body of the optical waveguide.

EEE 19 is the system of any of EEEs 1-18, wherein the first surface of the optical waveguide comprises one or more surface features configured to allow the portion of the illumination light to escape the optical waveguide from the first surface as the illumination light propagates toward the second end of the optical waveguide, and wherein the one or more surface features comprise diffractive features, longitudinal striations, lateral striations, isotropic striations, or pits.

EEE 20 is a system comprising:

a substrate having a first channel, a second channel, a third channel, a fourth channel, and a fifth channel defined therein, wherein the first channel is separated from the second channel by a first partial wall structure, wherein the second channel is separated from the third channel by a second partial wall structure, wherein the first channel is separated from the fourth channel by a third partial wall structure, and wherein the fourth channel is separated from the fifth channel by a fourth partial wall structure;

a first optical waveguide configured to:
receive first illumination light at a first end of the first optical waveguide;
propagate the first illumination light toward a second end of the first optical waveguide;
allow at least a portion of the first illumination light to escape the first optical waveguide from a first surface of the first optical waveguide as the first illumination light propagates toward the second end of the first optical waveguide; and
provide the portion of the first illumination light that escapes the first optical waveguide from the first surface to the second channel or the third channel; and

a second optical waveguide configured to:
receive second illumination light at a first end of the second optical waveguide;
propagate the second illumination light toward a second end of the second optical waveguide;
allow at least a portion of the second illumination light to escape the second optical waveguide from a first surface of the second optical waveguide as the second illumination light propagates toward the second end of the second optical waveguide; and
provide the portion of the second illumination light that escapes the second optical waveguide from the first surface of the second optical waveguide to the fourth channel or the fifth channel.

EEE 21 is the system of EEE 20, wherein the first optical waveguide is suspended above the second channel or the third channel, and wherein the second optical waveguide is suspended above the fourth channel or the fifth channel.

EEE 22 is the system of EEE 20, wherein the first optical waveguide is positioned on the substrate adjacent to the third channel or overlays the second channel or the third channel, and wherein the second optical waveguide is positioned on the substrate adjacent to the fifth channel or overlays the fourth channel or the fifth channel.

EEE 23 is a system comprising:

a plurality of substrates arranged into a vertical stack, wherein each of the substrates comprises a first channel, a second channel, a third channel, a fourth channel, and a fifth channel defined therein, wherein the first channel of each substrate is separated from the second channel of each substrate by a first partial wall structure, wherein the second channel of each substrate is separated from the third channel of each substrate by a second partial wall structure, wherein the first channel of each substrate is separated from the fourth channel of each substrate by a third partial wall structure, and wherein the fourth channel of each substrate is separated from the fifth channel of each substrate by a fourth partial wall structure;

a first optical waveguide configured to:
receive first illumination light at a first end of the first optical waveguide;
propagate the first illumination light toward a second end of the first optical waveguide;
allow at least a portion of the first illumination light to escape the first optical waveguide from a first surface of the first optical waveguide as the first illumination light propagates toward the second end of the first optical waveguide; and
provide the portion of the first illumination light that escapes the first optical waveguide from the first surface to the second channels or the third channels defined within each of the plurality of substrates; and

a second optical waveguide configured to:
receive second illumination light at a first end of the second optical waveguide;
propagate the second illumination light toward a second end of the second optical waveguide;
allow at least a portion of the second illumination light to escape the second optical waveguide from a first surface of the second optical waveguide as the second illumination light propagates toward the second end of the second optical waveguide; and
provide the portion of the second illumination light that escapes the second optical waveguide from the first surface of the second optical waveguide to the fourth channels or the fifth channels defined within each of the plurality of substrates.

EEE 24 is a method comprising:

infusing a cellular precursor into a second channel defined within a substrate;

administering liquid nutrient media into a first channel defined within the substrate, wherein the first channel is separated from the second channel by a first partial wall structure;

providing a first set of environmental conditions, wherein the first set of environmental conditions results in a growth of cells within the cellular precursor;

providing a second set of environmental conditions, wherein the second set of environmental conditions results in a production of trichomes by the cells within the cellular precursor, and wherein providing the first set of environmental conditions or providing the second set of environmental conditions comprises:
receiving, at a first end of an optical waveguide, illumination light;
propagating the illumination light toward a second end of the optical waveguide;
allowing at least a portion of the illumination light to escape the optical waveguide from a first surface of the optical waveguide as the illumination light propagates toward the second end of the optical waveguide; and
providing the portion of the illumination light that escapes the optical waveguide from the first surface to the second channel or a third channel defined within the substrate, wherein the second channel is separated from the third channel by a second partial wall structure; and

harvesting one or more of the produced trichomes or one or more chemical products contained within the produced trichomes.

EEE 25 is the method of EEE 24, further comprising performing a sterilization procedure, wherein the sterilization procedure comprises:

heating the substrate or the optical waveguide to a first predetermined temperature for a first predetermined time period; and

cooling the substrate or the optical waveguide to a second predetermined temperature.

EEE 26 is the method of EEE 25, wherein first predetermined temperature is between 175° C. and 185° C., and wherein the second predetermined temperature is between 20° C. and 25° C.

EEE 27 is the method of any of EEEs 24-26, further comprising monitoring the growth of cells within the cellular precursor or monitoring the production of trichomes by the cells within the cellular precursor using one or more imaging modalities, wherein the one or more imaging modalities comprise:

capturing one or more red-green-blue (RGB) images using an optical microscope;

performing hyperspectral imaging;

performing Raman spectroscopy; or

performing fluorescence spectroscopy.

EEE 28 is the method of any of EEEs 24-27,

wherein the cellular precursor comprises a gel precursor media,

wherein a gel of the gel precursor media comprises agar gel, agarose gel, alginate gel, gelatin gel, acrylamide gel, silica gel, or cellulose gel, and

wherein the cells within the cellular precursor comprise protoplast cell cultures, suspension cell cultures, or micro-calli cell cultures.

EEE 29 is the method of EEE 28, wherein infusing the cellular precursor comprises waiting a predetermined amount of time for the gel of the gel precursor media to solidify, and wherein the predetermined amount of time is between 0.25 hours and 2.0 hours.

EEE 30 is the method of any of EEEs 24-29, wherein providing the first set of environmental conditions comprises:

providing a temperature of between 20° C. and 25° C. to the second channel or the third channel;

providing the portion of the illumination light that escapes the optical waveguide from the first surface to the second channel or third channel for about 16 hours;

providing no illumination light to the second channel or third channel for about 8 hours; and

providing a calli-induction media into the first channel.

EEE 31 is the method of any of EEEs 24-30, wherein providing the second set of environmental conditions comprises:

providing a temperature of between 20° C. and 25° C. to the second channel or the third channel;

providing the portion of the illumination light that escapes the optical waveguide from the first surface to the second channel or third channel for about 12 hours;

providing no illumination light to the second channel or third channel for about 12 hours; and

providing a trichome-induction media into the first channel.

EEE 32 is the method of any of EEEs 24-31, wherein harvesting the one or more trichomes or the one or more chemical products contained within the produced trichomes comprises:

providing a temperature of less than 4° C. to the second channel or the third channel;

providing no illumination light to the second channel or third channel; and

flowing low-temperature extractant through the third channel at a sufficient flow rate so as to shear the one or more trichomes from the cells within the cellular precursor.

EEE 33 is the method of EEE 32, wherein the low-temperature extractant comprises ethanol at a temperature of between −75° C. and 0° C.

EEE 34 is a system comprising:

a substrate having a first channel, a second channel, and a third channel defined therein, wherein the first channel is separated from the second channel by a first partial wall structure, and wherein the second channel is separated from the third channel by a second partial wall structure; and

an optical waveguide configured to:
receive illumination light at a first end of the optical waveguide;
propagate the illumination light toward a second end of the optical waveguide;
allow at least a portion of the illumination light to escape the optical waveguide from a first surface of the optical waveguide as the illumination light propagates toward the second end of the optical waveguide; and
provide the portion of the illumination light that escapes the optical waveguide from the first surface to at least one of the second channel or the third channel.

What is claimed:

1. A system comprising:
a substrate having a first channel, a second channel, and a third channel defined therein, wherein the first channel is separated from the second channel by a first partial wall structure, and wherein the second channel is separated from the third channel by a second partial wall structure; and
an optical waveguide configured to:
receive illumination light at a first end of the optical waveguide;
propagate the illumination light toward a second end of the optical waveguide;
allow at least a portion of the illumination light to escape the optical waveguide from a first surface of the optical waveguide as the illumination light propagates toward the second end of the optical waveguide; and
provide the portion of the illumination light that escapes the optical waveguide from the first surface to the second channel or the third channel.

2. The system of claim 1,
wherein the second channel is figured to house parenchymal cells suspended in gel,
wherein the first channel is configured to supply liquid nutrient media to the parenchymal cells held in the second channel, and
wherein the third channel is configured to house trichomes produced by the parenchymal cells.

3. The system of claim 2,
wherein the first channel comprises a first inlet and a first outlet,
wherein the first inlet and the first outlet permit fluid communication between the first channel and an exterior of the system,
wherein the second channel comprises a second inlet and a second outlet,
wherein the second inlet and the second outlet permit fluid communication between the second channel and the exterior of the system,
wherein the third channel comprises a third inlet and a third outlet, and
wherein the third inlet and the third outlet permit fluid communication between the third channel and the exterior of the system.

4. The system of claim 2, wherein the parenchymal cells comprise Cannabis sativa cells, Artemisia annua cells, Chrysanthemum cinerariifolium cells, Chrysanthemum coccineum cells, Gossypium hirsutum cells, Gossypium barbadense cells, Gossypium arboreum cells, Gossypium herbaceum cells, Lavandula angustifolia cells, Arabidopsis thaliana cells, Mentha x piperita cells, or Mentha haplocalyx cells.

5. The system of claim 2, wherein the trichomes comprise cannabidiol, tetrahydrocannabinol, artemisinin, pyrethrum, camphor, glucosinolate, linalool, linalyl acetate, menthol, or peppermint camphor.

6. The system of claim 2, wherein the liquid nutrient media comprise an inorganic salt, a carbon source, myoinositol, glycine, a vitamin, a growth regulator, a nitrogen compound, an organic acid, or a plant extract.

7. The system of claim 2, wherein the third channel is configured to be infused with an extractant in order to harvest the trichomes produced by the parenchymal cells.

8. The system of claim 1, wherein the substrate has a fourth channel and a fifth channel defined there, wherein the first channel is separated from the fourth channel by a third partial wall structure, wherein the fourth channel is separated from the fifth channel by a fourth partial wall structure, and wherein the system further comprises an additional optical waveguide configured to:
receive additional illumination light at a first end of the additional optical waveguide;
propagate the additional illumination light toward a second end of the additional optical waveguide;
allow at least a portion of the additional illumination light to escape the additional optical waveguide from a first surface of the additional optical waveguide as the additional illumination light propagates toward the second end of the additional optical waveguide; and
provide the portion of the additional illumination light that escapes the additional optical waveguide from the first surface of the additional optical waveguide to the fourth channel or the fifth channel.

9. The system of claim 1, further comprising:
a second substrate having a fourth channel, a fifth channel, and a sixth channel defined therein,
wherein the fourth channel is separated from the fifth channel by a third partial wall structure, and wherein the fifth channel is separated from the sixth channel by a fourth partial wall structure, and
wherein the optical waveguide is further configured to provide the portion of the illumination light that escapes the optical waveguide from the first surface of the optical waveguide to the fifth channel or the sixth channel.

10. The system of claim 9, wherein the second substrate is positioned vertically adjacent to the substrate.

11. The system of claim 1,
wherein the substrate has a fourth channel, a fifth channel, and a sixth channel defined therein,
wherein the fourth channel is separated from the fifth channel by a third partial wall structure,
wherein the fifth channel is separated from the sixth channel by a fourth partial wall structure,
wherein the fourth channel, the fifth channel, and the sixth channel are substantially parallel to the first channel, the second channel, and the third channel, respectively, and
wherein the system further comprises an additional optical waveguide configured to:
receive illumination light at a first end of the additional optical waveguide;
propagate the illumination light toward a second end of the additional optical waveguide;
allow at least a portion of the illumination light to escape the additional optical waveguide from a first surface of the additional optical waveguide as the illumination light propagates toward the second end of the additional optical waveguide; and
provide the portion of the illumination light that escapes the additional optical waveguide from the first surface of the additional optical waveguide to the fifth channel or the sixth channel.

12. The system of claim 11, wherein the third channel and the sixth channel are defined within the substrate adjacently to one another with a spacing therebetween, and wherein the spacing is sufficient to permit imaging of one or more substances contained within the third channel or the sixth channel.

13. The system of claim 1, wherein the optical waveguide is suspended above the second channel or the third channel.

14. The system of claim 1, wherein the optical waveguide is positioned on the substrate adjacent to the third channel or overlays the second channel or the third channel.

15. The system of claim 1, wherein the optical waveguide is configured such that the portion of the illumination light that escapes the optical waveguide from the first surface and is provided to the second channel or the third channel is of substantially uniform intensity along a length direction of the second channel or a length direction of the third channel.

16. The system of claim 1, wherein the optical waveguide is configured such that the portion of the illumination light that escapes the optical waveguide from the first surface and is provided to the second channel or the third channel has an intensity that varies along a length direction of the second channel or a length direction of the third channel.

17. The system of claim 1, wherein the optical waveguide is tapered in at least one dimension between the first end and the second end.

18. The system of claim 1, wherein the optical waveguide comprises:
a mixing region configured to homogenize modes or wavelengths present within the illumination light received at the first end of the optical waveguide; and
a coupling region configured to couple the homogenized illumination light from the mixing region into a main body of the optical waveguide.

19. The system of claim 1, wherein the first surface of the optical waveguide comprises one or more surface features configured to allow the portion of the illumination light to escape the optical waveguide from the first surface as the illumination light propagates toward the second end of the optical waveguide, and wherein the one or more surface features comprise diffractive features, longitudinal striations, lateral striations, isotropic striations, or pits.

20. A system comprising:
a plurality of substrates arranged into a vertical stack, wherein each of the substrates comprises a first channel, a second channel, a third channel, a fourth channel, and a fifth channel defined therein, wherein the first channel of each substrate is separated from the second channel of each substrate by a first partial wall structure, wherein the second channel of each substrate is separated from the third channel of each substrate by a second partial wall structure, wherein the first channel of each substrate is separated from the fourth channel of each substrate by a third partial wall structure, and wherein the fourth channel of each substrate is separated from the fifth channel of each substrate by a fourth partial wall structure;
a first optical waveguide configured to:
receive first illumination light at a first end of the first optical waveguide;
propagate the first illumination light toward a second end of the first optical waveguide;
allow at least a portion of the first illumination light to escape the first optical waveguide from a first surface of the first optical waveguide as the first illumination light propagates toward the second end of the first optical waveguide; and
provide the portion of the first illumination light that escapes the first optical waveguide from the first surface to the second channels or the third channels defined within each of the plurality of substrates; and
a second optical waveguide configured to:
receive second illumination light at a first end of the second optical waveguide;
propagate the second illumination light toward a second end of the second optical waveguide;
allow at least a portion of the second illumination light to escape the second optical waveguide from a first surface of the second optical waveguide as the second illumination light propagates toward the second end of the second optical waveguide; and
provide the portion of the second illumination light that escapes the second optical waveguide from the first surface of the second optical waveguide to the fourth channels or the fifth channels defined within each of the plurality of substrates.

21. A method comprising:
infusing a cellular precursor into a second channel defined within a substrate;
administering liquid nutrient media into a first channel defined within the substrate, wherein the first channel is separated from the second channel by a first partial wall structure;
providing a first set of environmental conditions, wherein the first set of environmental conditions results in a growth of cells within the cellular precursor;
providing a second set of environmental conditions, wherein the second set of environmental conditions results in a production of trichomes by the cells within the cellular precursor, and wherein providing the first set of environmental conditions or providing the second set of environmental conditions comprises:
receiving, at a first end of an optical waveguide, illumination light;
propagating the illumination light toward a second end of the optical waveguide;
allowing at least a portion of the illumination light to escape the optical waveguide from a first surface of the optical waveguide as the illumination light propagates toward the second end of the optical waveguide; and
providing the portion of the illumination light that escapes the optical waveguide from the first surface to the second channel or a third channel defined within the substrate, wherein the second channel is separated from the third channel by a second partial wall structure; and
harvesting one or more of the produced trichomes or one or more chemical products contained within the produced trichomes.

22. The method of claim 21, further comprising performing a sterilization procedure, wherein the sterilization procedure comprises:
heating the substrate or the optical waveguide to a first predetermined temperature for a first predetermined time period; and
cooling the substrate or the optical waveguide to a second predetermined temperature.

23. The method of claim 22, wherein the first predetermined temperature is between 175° C. and 185° C., and wherein the second predetermined temperature is between 20° C. and 25° C.

24. The method of claim 21, further comprising monitoring the growth of cells within the cellular precursor or monitoring the production of trichomes by the cells within the cellular precursor using one or more imaging modalities, wherein the one or more imaging modalities comprise:
capturing one or more red-green-blue (RGB) images using an optical microscope;
performing hyperspectral imaging;
performing Raman spectroscopy; or
performing fluorescence spectroscopy.

25. The method of claim 21,
wherein the cellular precursor comprises a gel precursor media,
wherein a gel of the gel precursor media comprises agar gel, agarose gel, alginate gel, gelatin gel, acrylamide gel, silica gel, or cellulose gel, and
wherein the cells within the cellular precursor comprise protoplast cell cultures, suspension cell cultures, or micro-calli cell cultures.

26. The method of claim 25, wherein infusing the cellular precursor comprises waiting a predetermined amount of time for the gel of the gel precursor media to solidify, and wherein the predetermined amount of time is between 0.25 hours and 2.0 hours.

27. The method of claim 21, wherein providing the first set of environmental conditions comprises:
providing a temperature of between 20° C. and 25° C. to the second channel or the third channel;
providing the portion of the illumination light that escapes the optical waveguide from the first surface to the second channel or third channel for about 16 hours;
providing no illumination light to the second channel or third channel for about 8 hours; and
providing a calli-induction media into the first channel.

28. The method of claim 21, wherein providing the second set of environmental conditions comprises:
providing a temperature of between 20° C. and 25° C. to the second channel or the third channel;
providing the portion of the illumination light that escapes the optical waveguide from the first surface to the second channel or third channel for about 12 hours;
providing no illumination light to the second channel or third channel for about 12 hours; and
providing a trichome-induction media into the first channel.

29. The method of claim 21, wherein harvesting the one or more trichomes or the one or more chemical products contained within the produced trichomes comprises:
providing a temperature of less than 4° C. to the second channel or the third channel;
providing no illumination light to the second channel or third channel; and
flowing low-temperature extractant through the third channel at a sufficient flow rate so as to shear the one or more trichomes from the cells within the cellular precursor.

30. The method of claim 29, wherein the low-temperature extractant comprises ethanol at a temperature of between −75° C. and 0° C.
